(19) United States
(12) Patent Application Publication
(10) Pub. No.: US 2026/0240703 A1
(43) Pub. Date: Aug. 20, 2026

(54) PATIENT TRANSPORTATION ASSISTANCE AND CONTROL

(71) Applicant: Able Innovations Inc., Toronto (CA)

(72) Inventors: Philip Chang; Bahareh Chimehi; Jian Huang; Nara Levi; Ryan Wang; Bruce Wallace

(21) Appl. No.: 19/470,724

(22) PCT Filed: Mar. 28, 2024
(86) PCT No.: PCT/CA2024/050414

§ 371 Date: Sep. 29, 2025

Related U.S. Application Data
(60) Provisional application No. 63/456,132, filed on Mar. 31, 2023.

(51) Int. Cl. A61G 7/08; A61G 12/00; G05D 1/622; G05D 105/22; G16H 40/63

(52) U.S. Cl. CPC A61G 7/08; A61G 12/001; G05D 1/622; G16H 40/63; A61G 2203/32; G05D 2105/22

(57) ABSTRACT

A healthcare facility transport device includes a base with wheels including a powered wheel, a carrying structure, first and second handles, first and second force sensors, and a control system comprising a system processor. The carrying structure is configured to carry at least one of healthcare supplies and a patient. The first and second force sensors are mounted proximate to the first and second handles, respectively, and are configured to output respective first and second force signals in response to sensing movement of the first and second handles, respectively. The control system is configured to determine a magnitude and direction of an updated velocity vector and operate the powered wheel with the updated velocity vector. In various cases the updated velocity vector is based on the force signal values, a first model, a current velocity, and/or a distance between the first and second handles.

### CROSS-REFERENCE TO RELATED APPLICATION(S)

This application claims the benefit under 35 U.S.C. § 119(e) to U.S. Provisional Application 63/456,132, filed Mar. 31, 2023, and entitled “Patient Transfer Device With Transportation Assistant,” which is hereby incorporated herein by reference in its entirety.

### TECHNICAL FIELD

The disclosed technology relates generally to devices for moving objects, including patients, within healthcare facilities, and more particularly to powered transportation devices and control systems associated thereof.

### BACKGROUND

In healthcare facilities worldwide, caregivers frequently move immobile patients for many reasons, such as preparing for various medical procedures or simply to change bed sheets. Such movements include transferring a patient from one surface to another (e.g., from a bed to a gurney) and transporting a patient to various places in a healthcare facility such as a hospital or residential care facility. For example, a patient may be transported from a hospital care room to a diagnostic imaging facility.

Currently, the majority of patient transfers are performed manually by anywhere from two to eight people or with the assistance of a mechanical apparatus such as a transfer board or sling system assisted by either an overhead lift or mobile floor lift. Sling-based lifts have been shown to be uncomfortable and unsafe for patients and on occasion have caused injury and even death from falls under extreme circumstances. For staff, many workers can be needed to safely move a patient onto and off of the sling depending on the patient's weight. This physical work puts the medical workers at risk of injury. As a result, nursing and other healthcare personnel have very high rates of musculoskeletal injuries.

Conveyor-based patient transfer devices have been developed in order to reduce the burden and repetitive strain on healthcare providers associated with patient transfers and transportation operations. These new conveyor-based patient transfer and transportation devices have also been shown to exert less strain and discomfort to the patient as well as to provide an overall more pleasant and dignified transfer and transportation experience. While these new devices are more efficient and less burdensome to use for patient transfers, the devices can be heavy and difficult to move due to the weight of the mechatronics and battery required for their function. In addition, maneuvering these large and heavy devices through narrow corridors that are often busy and filled with staff, patients, and medical equipment can be difficult, with small maneuvers requiring significant effort. As a result, transporting a patient or other object on these types of medical devices can be cumbersome and also induce injuries, strain, or additional fatigue. Such difficulties can also occur with other types of medical conveyances and furniture such as wheeled stretchers and hospital beds. Some powered transport stretchers and gurneys do exist, but they most often implement limited user input methods and assistance capabilities. This often leads to injuries and as such requires a minimum of two operators to perform a transportation operation. Similar challenges exist with the movement of heavily laden medical supply carts, often leading to fatigue and repetitive injuries.

### SUMMARY

The disclosed technology provides healthcare facility transportation devices and related control systems, devices and methods for moving or transporting an object (e.g., a patient) from place to place within a healthcare facility. The disclosed technology further provides methods for training a machine learning algorithm useful for controlling the transportation device. According to one aspect, the disclosed technology relates to healthcare facility transportation devices and associated control systems and methods for moving objects about a healthcare facility. According to another aspect, the disclosed technology relates to patient transportation devices. According to another aspect, example transportation devices generate an updated velocity vector for operating a powered wheel of the transportation device. In various cases the updated velocity vector is determined based on an operator's use of one or more drive controls, a distance between the drive controls, a first model, and a current velocity of the transport device. In various cases the first model is based on a finite-state machine or a machine learning model. Methods for training a machine learning algorithm for controlling such healthcare facility and patient transportation devices are also provided according to another aspect of the technology.

Various implementations of the disclosed technology include a system of one or more computers that are configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Examples of various implementations of the disclosed technology include, but are not limited to, the following:
In Example 1, a healthcare facility transport device, comprising: a base comprising a plurality of wheels comprising a powered wheel, a carrying structure mounted to the base and configured to carry at least one of healthcare supplies and a patient, first and second handles mounted to the carrying structure, a first force sensor mounted proximate to the first handle and configured to output a first force signal in response to sensing movement of the first handle, a second force sensor mounted proximate to the second handle and configured to output a second force signal in response to sensing movement of the second handle, and a control system comprising a system processor configured to: determine an updated velocity vector, comprising: determining a magnitude of the updated velocity vector based on values of the first and second force signals, and determining a direction of the updated velocity vector based on the values of the first and second force signals, a first model, a current velocity of the transport device, and a distance between the first and second handles, and operate the powered wheel based on the updated velocity vector.
In Example 2, the healthcare facility transport device of Example 1, wherein the first model comprises a first finite-state machine.
In Example 3, the healthcare facility transport device of Example 1, wherein the first model comprises a first machine learning model.
In Example 4, the healthcare facility transport device of Example 3, wherein the first machine learning model is a product of a decision tree learning algorithm.
In Example 5, the healthcare facility transport device of Example 3, wherein the first machine learning model is a product of a random forest learning algorithm.
In Example 6, the healthcare facility transport device of any one of Examples 1 to 5, wherein the system processor is further configured to determine augmentation values for the updated velocity vector angle and magnitude based on the current velocity.
In Example 7, the healthcare facility transport device of any one of Examples 1 to 5, wherein the system processor is further configured to determine the velocity vector based on a second machine learning model.
In Example 8, the healthcare facility transport device of any one of Examples 1 to 7, further comprising a first plurality of force sensors mounted proximate to the first handle, each of the first plurality of force sensors configured to output a respective one of a first plurality of force signals in response to sensing movement of the first handle, the first plurality of force sensors comprising: the first force sensor and a third force sensor arranged to sense movement of the first handle, respectively, in a first direction and a third direction opposite the first direction, and fourth and fifth force sensors arranged to sense movement of the first handle, respectively, in a fourth direction and a fifth direction opposite the fourth direction, and a second plurality of force sensors mounted proximate to the second handle, each of the second plurality of force sensors configured to output a respective one of a second plurality of force signals in response to sensing movement of the second handle, the second plurality of force sensors comprising: the second force sensor and a sixth force sensor arranged to sense movement of the second handle, respectively, in a second direction and a sixth direction opposite the second direction, and seventh and eighth force sensors arranged to sense movement of the second handle, respectively, in a seventh direction and an eighth direction opposite the seventh direction.
In Example 9, the healthcare facility transport device of Example 8, wherein the first plurality of force signals comprises the first force signal and third, fourth, and fifth force signals respectively corresponding to the first, third, fourth and fifth force sensors, and wherein the velocity vector is based on a Pythagorean sum and trigonometric calculation of a difference in values of the first and third force signals and a difference in values of the fourth and fifth force signals.
In Example 10, the healthcare facility transport device of Example 9, wherein the first and third directions are orthogonal with the fourth and fifth directions, and the second and sixth directions are orthogonal with the seventh and eighth directions.
In Example 11, the healthcare facility transport device of any one of Examples 8 to 10, wherein the first and third force sensors are symmetrically arranged about the first handle, the fourth and fifth force sensors are symmetrically arranged about the first handle, the second and sixth force sensors are symmetrically arranged about the second handle, and the seventh and eighth force sensors are symmetrically arranged about the second handle.
In Example 12, the healthcare facility transport device of any one of Examples 1 to 11, wherein the first and second handles are flexibly mounted to the carrying structure, wherein the first force sensor is connected between the first handle and the carrying structure, and wherein the second force sensor is connected between the second handle and the carrying structure.
In Example 13, the healthcare facility transport device of any one of Examples 1 to 12, further comprising: a first handle mount assembly comprising: a first plate attached to the carrying structure, a first lever arm connected to and extending away from the first plate, and a second handle mount assembly comprising: a second plate attached to the carrying structure, a second lever arm connected to and extending away from the second plate, wherein the first handle is connected to the first plate, wherein the second handle is connected to the second plate, wherein a first end of the first sensor is connected to the first lever arm and a second end of the first sensor is connected to the carrying structure, and wherein a first end of the second sensor is connected to the second lever arm and a second end of the second sensor is connected to the carrying structure.
In Example 14, the healthcare facility transport device of any one of Examples 1 to 13, wherein the system processor is further configured to augment the velocity vector based on a mass factor.
In Example 15, the healthcare facility transport device of any one of Examples 1 to 14, further comprising at least one of an inertial measurement unit (IMU) and an encoder, wherein the system processor is further configured to determine a measured velocity vector of the healthcare facility transport device based on an output signal from the IMU and/or encoder.
In Example 16, the healthcare facility transport device of Examples 14 or 15, wherein the system processor is configured to increase the mass factor if the velocity vector opposes the measured current velocity.
In Example 17, the healthcare facility transport device of any one of Examples 14 to 16, wherein the system processor is configured to increase the mass factor to simulate decreased inertia if a magnitude of the measured velocity is below a low-speed threshold.
In Example 18, the healthcare facility transport device of any one of Examples 14 to 17, wherein the system processor is configured to decrease the mass factor to simulate increased inertia if a magnitude of the measured velocity is above a high-speed threshold.
In Example 19, the healthcare facility transport device of any one of Examples 1 to 18, wherein the first handle comprises a first presence sensor and the second handle comprises a second presence sensor.
In Example 20, the healthcare facility transport device of any one of Examples 1 to 19, wherein the first and second handles are mounted at an end of the carrying structure.
In Example 21, the healthcare facility transport device of Example 20, wherein the first and second handles extend at least partially along a width of the carrying structure.
In Example 22, the healthcare facility transport device of any one of Examples 1 to 19, wherein the first and second handles are mounted at a side of the carrying structure and extend at least partially along a length of the carrying structure.
In Example 23, the healthcare facility transport device of any one of Examples 1 to 22, wherein the transport device is a medical cart.
In Example 24, the healthcare facility transport device of any one of Examples 1 to 22, wherein the transport device is a patient transfer device.
In Example 25, a patient transport device, comprising: a transport base comprising a plurality of wheels comprising a powered wheel, a patient support structure mounted to the transport base, a first handle mounted to the patient support structure, a second handle mounted to the patient support structure, a first force sensor mounted proximate to the first handle and configured to output a first force signal in response to sensing movement of the first handle, the first force signal comprising a first force value, a second force sensor mounted proximate to the second handle and configured to output a second force signal in response to sensing movement of the second handle, the second force signal comprising a second force value, and a distributed control system comprising a system processor and a plurality of subsystem controllers, wherein each subsystem controller is configured to control operation of one of a plurality of subsystems of the patient transport device, and wherein the system processor is configured to: monitor and communicate with the plurality of subsystem controllers, determine an updated velocity vector, comprising: determining a magnitude of the updated velocity vector based on the first force value and the second force value, and determining a direction of the updated velocity vector based on the first force value, the second force value, a first model, a current velocity of the transport device, and a distance between the first and second handles, and operate the powered wheel based on the updated velocity vector.
In Example 26, the patient transport device of Example 25, further comprising: a first sensor assembly comprising a first plurality of force sensors mounted proximate to the first handle and comprising the first force sensor, each of the first plurality of force sensors configured to output a respective one of a first plurality of force signals, comprising the first force signal, in response to sensing movement of the first handle, the first plurality of force signals comprising, respectively, a first plurality of force values comprising the first force value, and a second sensor assembly comprising a second plurality of force sensors mounted proximate to the second handle and comprising the second force sensor, each of the second plurality of force sensors configured to output a respective one of a second plurality of force signals, comprising the second force signal, in response to sensing movement of the second handle, the second plurality of force signals comprising, respectively, a second plurality of force values comprising the second force value, wherein the system processor is further configured to determine the magnitude of the updated velocity vector based on the first and second pluralities of force values and determine the direction of the updated velocity vector based on the first and second pluralities of force values.
In Example 27, the patient transport device of any one of Examples 25 and 26, wherein the plurality of subsystems comprises a transfer subsystem comprising a transfer platform supported by the patient support structure, the transfer platform comprising a conveyor belt configured to move a patient onto and off of the transfer platform, and wherein the plurality of subsystem controllers comprises a transfer controller configured to operate the transfer subsystem.
In Example 28, the patient transport device of any one of Examples 25 to 27, wherein the plurality of subsystem controllers comprises a transport controller configured to operate the powered wheel based on the updated velocity vector.
In Example 29, the patient transport device of any one of Examples 25 to 28, wherein the first model comprises a first finite-state machine.
In Example 30, the patient transport device of any one of Examples 25 to 28, wherein the first model comprises a first machine learning model.
In Example 31, the patient transport device of Example 30, wherein the first machine learning model is a product of a decision tree learning algorithm.
In Example 32, the patient transport device of Example 30, wherein the first machine learning model is a product of a random forest learning algorithm.
In Example 33, the patient transport device of any one of Examples 25 to 32, wherein the system processor is further configured to determine the magnitude of the updated velocity vector based on a second machine learning model.
In Example 34, the patient transport device of any one of Examples 25 to 32, wherein the second machine learning model is a product of a decision tree learning algorithm or a random forest learning algorithm.
In Example 35, the patient transport device of any one of Examples 25 to 34, wherein the first plurality of force values further comprises third, fourth, and fifth force values and wherein the updated velocity vector is based on a Pythagorean sum and trigonometric calculation of a difference of the first and third force values and a difference of the fourth and fifth force values.
In Example 36, the patient transport device of any one of Examples 25 to 35, wherein the system processor is further configured to augment the updated velocity vector based on a mass factor.
In Example 37, the patient transport device of any one of Examples 25 to 36, wherein the first and second handles are mounted at an end of the patient support structure.
In Example 38, the patient transport device of any one of Examples 25 to 37, wherein the first and second handles are mounted at an end of the patient support structure and extend at least partially along a width of the patient support structure.
In Example 39, the patient transport device of any one of Examples 25 to 38, wherein the first and second handles are mounted at a side of the patient support structure and extend at least partially along a length of the patient support structure.
In Example 40, the patient transport device of any one of Examples 25 to 39, further comprising an angle sensor configured to sense a current pitch angle of the patient transport device, wherein the system processor is configured to adjust the updated velocity vector based on the current pitch angle so that the patient transport device decelerates on a decline and accelerates on an incline.
In Example 41, the patient transport device of any one of Examples 25 to 40, further comprising a plurality of obstacle sensors configured to generate proximity measurements for obstacles near the patient transport device.
In Example 42, the patient transport device of Example 41, wherein the plurality of obstacle sensors comprises at least one of a lidar sensor and an ultrasonic sensor.
In Example 43, the patient transport device of any one of Examples 25 to 42, further comprising a physical bumper sensor configured to signal physical contact with an object.
In Example 44, the patient transport device of any one of Examples 25 to 43, further comprising a camera configured to generate a video stream for autonomous navigation of the patient transport device.
In Example 45, the patient transport device of any one of Examples 25 to 44, wherein the system processor is configured to retrieve location data for the patient transport device and use the location data to autonomously drive the patient transport device from a first location to a second location.
In Example 46, the patient transport device of Example 45, wherein the system processor is configured to autonomously avoid obstacles while driving from the first location to the second location.
In Example 47, the patient transport device of any one of Examples 26 to 46, wherein the first plurality of force sensors comprises: the first force sensor and a third force sensor arranged to sense movement of the first handle, respectively, in a first direction and a third direction opposite the first direction, and fourth and fifth force sensors arranged to sense movement of the first handle, respectively, in a fourth direction and a fifth direction opposite the fourth direction, and the second plurality of force sensors comprises: the second force sensor and a sixth force sensor arranged to sense movement of the second handle, respectively, in a second direction and a sixth direction opposite the second direction, and seventh and eighth force sensors arranged to sense movement of the second handle, respectively, in a seventh direction and an eighth direction opposite the seventh direction.
In Example 48, the patient transport device of Example 47, wherein the first and third directions are orthogonal with the fourth and fifth directions, and the second and sixth directions are orthogonal with the seventh and eighth directions.
In Example 49, the patient transport device of any one of Examples 25 to 48, wherein the first and second handles are flexibly mounted to the patient support structure, wherein the first force sensor is connected between the first handle and the patient support structure, and wherein the second force sensor is connected between the second handle and the patient support structure.
In Example 50, the patient transport device of any one of Examples 22 to 49, further comprising: a first handle mount assembly comprising: a first plate attached to the patient support structure, a first frame connected to and extending away from the first plate, and a second handle mount assembly comprising: a second plate attached to the patient support structure, a second frame connected to and extending away from the second plate, wherein the first handle is connected to the first plate, wherein the second handle is connected to the second plate, wherein a first end of the first sensor is connected to the first frame and a second end of the first sensor is connected to the patient support structure, and wherein a first end of the second sensor is connected to the second frame and a second end of the second sensor is connected to the patient support structure.
In Example 51, a method for training a machine learning algorithm for generating an updated velocity vector for a patient transport device, the method comprising: providing a patient transport device comprising: a non-powered, free-wheeling transport base, a patient support structure mounted to the transport base, a first handle mounted to the patient support structure, a second handle mounted to the patient support structure, a first force sensor mounted proximate to the first handle and configured to output a first force signal in response to sensing movement of the first handle, the first force signal comprising a first force value, a second force sensor mounted proximate to the second handle and configured to output a second force signal in response to sensing movement of the second handle, the second force signal comprising a second force value, an inertial measurement unit (IMU), and a control system comprising a system processor configured to determine a measured current velocity of the patient transport device based on an output signal from the IMU, pushing and/or pulling at least one of the first handle and the second handle to move the patient transport device, recording multiple instances of the first force value, the second force value, and the measured current velocity to generate training data, and applying a first machine learning algorithm to the training data to produce a first machine learning model configured to predict a direction of an intended velocity vector corresponding to forces imparted to the first handle and/or the second handle.
In Example 52, the method of Example 51, further comprising applying a second machine learning algorithm to the training data to produce a second machine learning model configured to predict a magnitude of the intended velocity vector.
In Example 53, the training method of any one of Examples 51 to 52, wherein at least one of the first machine learning algorithm and the second machine learning algorithm is a decision tree algorithm.
In Example 54, the training method of any one of Examples 51 to 52, wherein at least one of the first machine learning algorithm and the second machine learning algorithm is a random forest algorithm.
In Example 55, a patient transport device, comprising: a transport base comprising a plurality of wheels comprising a powered wheel, a vertical actuation mechanism comprising a mounting bracket attached to the powered wheel, a drive train engaged with the mounting bracket, and a handle rotatably coupled with the drive train, a patient support structure mounted to the transport base, a first handle mounted to the patient support structure, a second handle mounted to the patient support structure, a first force sensor mounted proximate to the first handle and configured to output a first force signal in response to sensing movement of the first handle, the first force signal comprising a first force value, a second force sensor mounted proximate to the second handle and configured to output a second force signal in response to sensing movement of the second handle, the second force signal comprising a second force value, and a control system comprising a system processor configured to: determine an updated velocity vector, comprising: determining a magnitude of the updated velocity vector based on a sum of the first and second force values, and determining a direction of the updated velocity vector based on the first force value, the second force value, a first model, a current velocity of the transport device, and a distance between the first and second handles, and operate the powered wheel based on the updated velocity vector.
In Example 56, the healthcare facility transport device of any of claims 1 to 24, wherein the plurality of wheels comprises a plurality of powered wheels.
In Example 57, the patient transport device of any of claims 25 to 50, wherein the plurality of wheels comprises a plurality of powered wheels.
In Example 58, the patient transport device of claim 55, wherein the plurality of wheels comprises a plurality of powered wheels.

Other embodiments of these Examples include corresponding computer systems, apparatus, and computer programs recorded on one or more computer storage devices, each configured to perform the actions of the methods.

While multiple implementations and aspects are disclosed, still other embodiments of the disclosure will become apparent to those skilled in the art from the following detailed description, which shows and describes illustrative embodiments of the disclosed apparatus, systems, and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

Embodiments will now be described with reference to the attached drawings.

FIGS. 1A-1F are perspective views of example patient transportation devices according to various implementations.

FIGS. 2A-2C are schematic diagrams of example patient transportation devices according to various implementations.

FIGS. 3A-3B are opposite end views of an example patient transportation device depicting a proximity sensor array according to various implementations.

FIG. 3C is a schematic joint field of view for the proximity sensor array of FIGS. 3A-3B according to various implementations.

FIGS. 4A-4C are various views of a handle mount assembly according to various implementations.

FIGS. 5A-5G are schematic top and side views of various sensor assemblies for use with handles of a patient transportation device according to various implementations.

FIG. 6 is an exploded isometric view of a bumper sensor assembly according to various implementations.

FIG. 7A-7E are various views of a wheel lift mechanism according to various implementations.

FIGS. 8A-8F are schematic bottom views of various configurations of powered wheels and non-powered wheels of a patient transportation device according to various implementations.

FIGS. 9A-9F are schematic diagrams of relationships between a user's input to the patient transportation device and a resulting velocity vector according to various implementations.

FIGS. 10A-10B are block diagrams illustrating examples of control systems for a healthcare facility transportation device according to various implementations.

FIGS. 11A-11B are flow diagrams of methods for training a machine learning algorithm for generating a velocity vector for a patient transportation device.

FIGS. 12A-12B are examples of state tables for a control system according to various implementations.

FIGS. 13A-C are flow diagrams of methods for controlling a healthcare facility transportation device according to various implementations.

FIG. 14 is a perspective view of a healthcare facility transportation device according to various implementations.

### DETAILED DESCRIPTION

It should be understood at the outset that although illustrative implementations of one or more embodiments of the present disclosure are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

An aspect of the disclosed technology provides devices, systems, and methods for moving or transporting an object from place to place within a healthcare facility. Examples of a healthcare facility include, but are not limited to, a hospital, a clinic, a diagnostic imaging facility, a residential care facility, and other such facilities that provide healthcare services. Various implementations according to this aspect are useful for transporting objects such as patients, healthcare supplies, medications, diagnostic equipment, and other objects found in healthcare facilities. Accordingly, this aspect of the disclosure is often described herein as providing healthcare facility transportation systems, devices, and methods. Various implementations are further described as providing a healthcare facility transportation device 100, which is also referred to herein as a healthcare facility transport device 100, or simply a transportation device 100, a transport device 100, and variations thereof, though it should be understood that this is for brevity and convenience, and is in no way intended to be limiting to any specific modality.

Examples of healthcare facility transportation devices 100 include, but are not limited to, specialty procedural carts, emergency crash carts, linen carts, medication carts, food carts, and the like. A subset of healthcare facility transportation devices involves transporting a patient, and thus various implementations herein refer to a healthcare facility transportation device 100 as a patient transportation device or a patient transport device. Examples of possible patient transport devices include, but are not limited to, medical conveyances and mobile furniture such as wheeled stretchers, gurneys, trolleys, carts, mobile hospital beds, mobile examination, and treatment tables, and the like. In various cases, a patient transportation device can include a specialized patient transportation gurney such as an x-ray, maternity, gynecology, or pediatric gurney.

As noted above, various implementations are described as providing a healthcare facility transportation or transport device 100, a patient transportation or transport device 100, or simply a transportation device 100, a transport device 100, and variations thereof. It should be understood that these terms are at times used for brevity and convenience, which is in no way intended to be limiting to any specific modality. In particular, the terms “transportation device” and “transport device” are specifically used herein to refer to any of the variously disclosed or contemplated healthcare facility transportation devices, including transportation devices for moving patients and/or other objects, unless otherwise explicitly noted or implied from the context and/or usage.

As discussed further herein, various implementations of the disclosed technology provide a patient transportation device, system, and/or method that incorporates a patient transfer device. The patient transfer device includes a transfer platform and one or more conveyors for moving a patient onto or off of the transfer platform and device. Once the patient is moved onto the transfer platform, the patient transfer device can be used to transport the patient to another location. While various implementations discussed herein involve a patient transportation device in the form of a transfer device, it should be noted that the disclosed technology is not limited to the specific examples illustrated herein, and that various aspects of the illustrated examples can be implemented in various other patient and healthcare facility transportation devices, systems, and methods encompassed by the technology.

Another aspect of the disclosed technology provides a healthcare facility transportation device that includes a carrying structure mounted to a powered transport base. As an example, a patient transportation device can include a patient support structure mounted to a powered transport base. The transportation device also includes a control system configured to control the transport base, a human-machine interface (HMI) that allows a user to operate the transportation device, and one or more drive controls. In various implementations the user's interaction with one or more drive controls such as a handle, handlebar, joystick, or other input device indicates to the control system how to operate the transport base to propel and/or steer the transportation device. The term “powered transportation assistance” is sometimes used herein to refer to this capability to self-drive the transportation device based on the user's interaction with various drive controls.

Examples of the disclosed transportation devices, systems and/or methods provide improved maneuverability in comparison to traditional, non-powered transportation devices. In various implementations the transportation device can optionally provide powered, omni-directional transportation assistance. In various cases the transport device is configured to implement one, two, or more speeds. For example, the control system can optionally be configured to operate the transport base at a slow speed with high-precision maneuvering. Slow-speed capabilities can be especially useful for moving large devices (e.g., wheeled hospital beds, etc.) through narrow spaces, around corners, along crowded hallways, and the like. In various cases the control system is optionally configured to provide a high-speed mode of operation. Such high-speed operation can enable various transport devices to more quickly traverse longer, open spaces, such as unoccupied hallways.

According to another aspect of the technology, various healthcare facility transportation devices, systems, and methods provide powered transportation assistance to a user in an intuitive manner, optionally without requiring exertion from the user. As an example, the transportation device may include one or more intuitive drive controls. Such drive controls can in some cases include one or more sensors coupled with one or more portions of the transportation device that a user naturally interacts with. For example, one or more sensors may be coupled with a push handle, a handlebar, a side rail, a foot pedal, or another part of the transportation device. The sensor(s) generate signal(s) (sometimes referred to herein as force signals) in response to the user's handling of that part of the transportation device. The control system receives this feedback from the drive control(s) and interprets a desired transportation direction and speed for moving the transport base. In various examples the transportation device is further configured with systems and methods for training and improving the operation of the control system.

The control system is thus able to move the transportation device based on a user's interaction with parts of the transportation device that will be familiar, natural, and intuitive to interact with. This intuitive manner of initiating self-propulsion of the transportation device also avoids the need for additional dedicated or special-purpose input devices. The use of such intuitive drive controls can thus reduce the need for additional training and shorten the time required for staff to develop familiarity with the system.

Accordingly, it will be appreciated that various implementations of the disclosed technology provide advantages over and/or address concerns present in existing transportation and powered assistant systems. As an example, in some cases the disclosed technology provides an intuitive user interface and drive control (e.g., for medical staff) that provides advantages over existing drive controls that feature unintuitive twist-grip type or thumb throttle user inputs for operation. In some cases implementations include drive control devices with sensors that are less exposed and/or less sensitive to aggressive cleaning cycles that are, e.g., dictated by environmental requirements.

As another example, various implementations provide a patient transfer device, incorporating the features of various aforementioned transportation devices which requires little to no user exertion. In some cases, these implementations provide advantages over existing powered transport solutions in which an operator must still generate enough energy to overcome the inertia of the device. This particularly poses a problem for any staff that may return to work on modified or reduced duties protocols. Often such staff are limited in how much effort they are allowed to exert (for example 2.5 to 5 kg). In such circumstances the static inertia of a heavy patient on a gurney or other transport device can exceed these limitations, rendering the powered transportation assistance inappropriate and perhaps even unsafe to use as they pose a risk for reinjury. Often, due to the complexity or unwieldly nature of even power-assisted transportation gurneys and stretchers there is a need for a minimum of two operators to ensure safe operation. Even in the case of powered stretchers, with simple or unintuitive drive controls, it can be difficult to maintain control over the power assisted device and perform simple or short-distance micro-adjustments in position or macro level maneuvers without two operators present. Various implementations of a transportation device according to the disclosed technology can significantly reduce or entirely eliminate the physical exertion required by the user to control the movement of a transportation device 100, and in some cases limit the required exertion below that of the requirements for staff under modified duties or return to work ‘no-lift’ and modified return to work duty protocols.

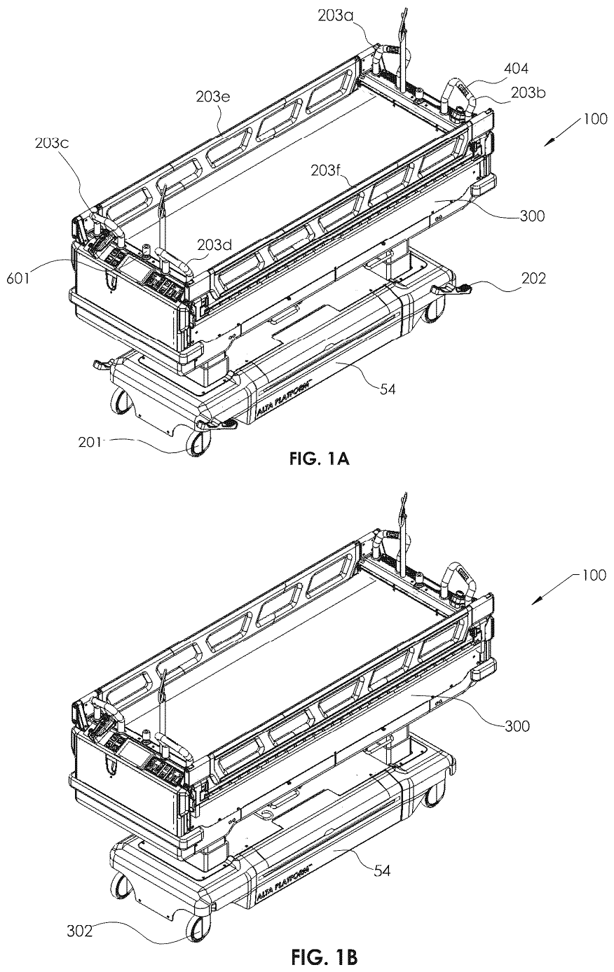

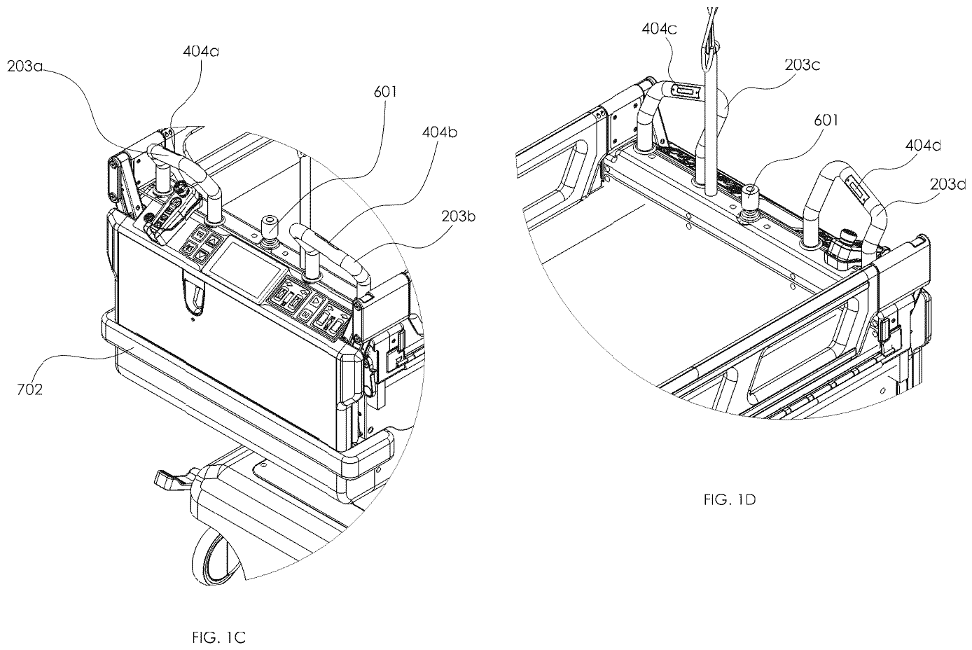

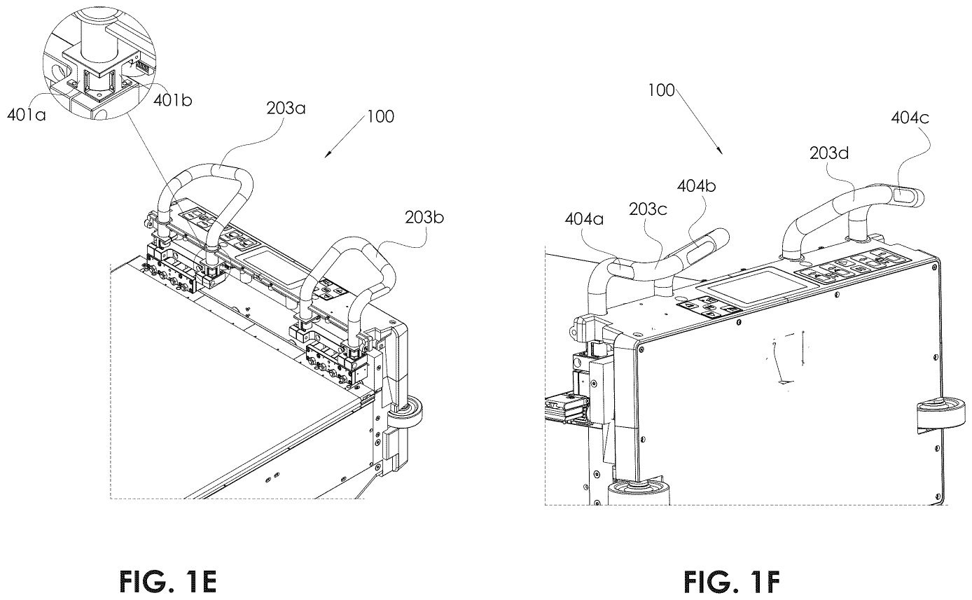

Turning now to the drawings, FIGS. 1A and 1B are perspective views of examples of patient transportation devices 100 according to various implementations of the disclosed technology. The transportation device 100 includes a powered transport base 54 that in this example includes multiple, optional non-powered (also referred to as non-driven) casters or wheels 201. Although not shown in this view, the transport base 54 also includes at least one driven or powered wheel. The transport device 100 also includes a carrying structure 300 mounted to the transport base 54. As shown in FIGS. 1A-1F, the carrying structure is a patient support structure 300 generally configured to support a patient upon the device 100. The patient support structure in this example is implemented as a patient transfer platform. Other implementations of a patient support structure can include beds, chairs, tables, and other structures configured to support a patient. In various implementations the carrying structure of a healthcare facility transportation device may include cabinets and other storage structures, trays, tables, equipment, and/or other structures configured to carry various objects in a healthcare facility.

FIGS. 1C and 1D illustrate a patient transport device 100 with multiple drive controls that include several handles implemented as force sensitive handle bars 203a-d, side rails 203e-f, and an omnidirectional joystick 601. As used herein, the term “handle” is specifically used herein to commonly refer to various types of drive controls that can be manipulated by hand. The handlebars 203a-d include touch sensitive input devices 404a-d and are arranged in pairs (e.g., one left and one right) at each end of the transportation device 100 in this arrangement. FIG. 1C also illustrates a force sensitive bumper 702 on the end of the transportation device 100. The force sensitive bumper assembly 700 provides feedback to the control system in response to a physical interaction (e.g., collision) with the bumper 702. In various cases the feedback includes both an indication of the interaction and a magnitude of a physical interaction. Aspects of the force sensitive bumper are further discussed with respect to FIG. 6.

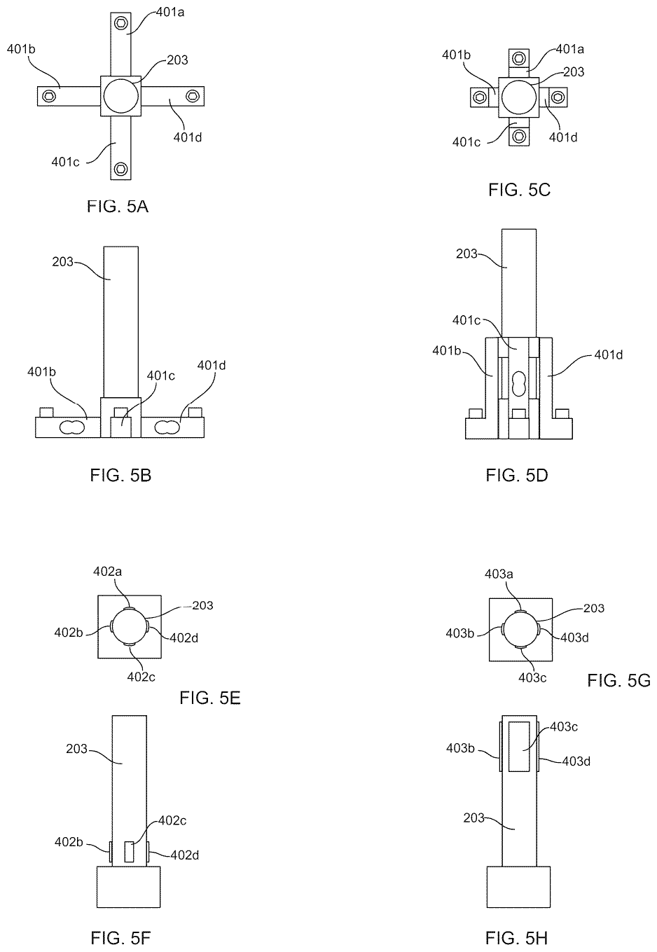

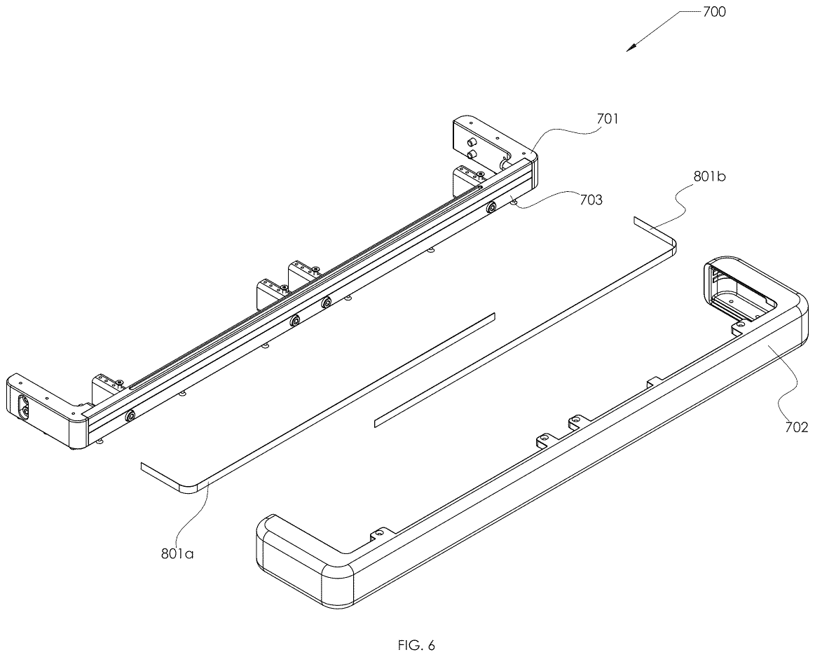

FIGS. 1E and 1F illustrate multiple drive controls 203 in the form of multi-axis force sensing handlebars (203a-d) that incorporate multi-axis touch sensing input devices (404a-c). In these implementations the drive controls 203 include multiple force sensors 401 that enable the drive controls to sense handle forces in both the longitudinal and transverse orientations with respect to the device frame. These components communicate with the control system for the transport device, which receives and uses feedback from the drive controls to determine a speed and direction for propelling the patient transfer device 100. Further aspects and details of various implementations of such multi-axis drive controls are discussed herein with respect to FIGS. 5A-5H.

Examples of the transportation device 100 may include various features and components depending upon the particular implementation and context. As examples, in some cases the transport device 100 has one or more free-wheeling (e.g., non-powered) casters along with one or more powered wheels. Alternative implementations may only include one or more powered wheels. In some cases the device 100 includes a pedal-based central locking system 202 which has two or more positions including, for example, one which is a complete lock in all directions. As noted above, in various cases the device 100 includes multiple drive controls, though it is contemplated that a single drive control could also be appropriate in some contexts. In various implementations the drive controls include sensors incorporated into handlebars 203a-d or user-interactive rails 203e-f (e.g., patient side rails 203e-f) that sense the presence of a user (e.g., touching the surfaces) and/or sense forces the user is exerting on the transport device 100 in efforts to perform transportation actions.

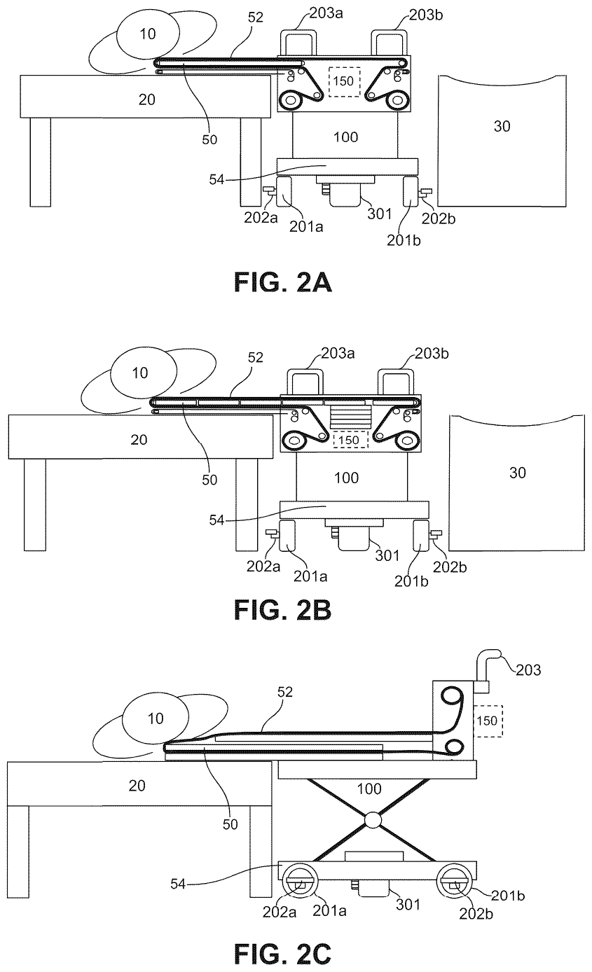

FIGS. 2A-2C illustrate various examples of a patient transport device 100 implemented as a patient transfer device, capable of moving a patient from one surface to another. In various implementations the patient transfer device generally includes one or more transfer platforms 50 used to support the weight of a patient 10 during transfer and transport operations. Examples of transfer operations include moving patients laterally into and out of a hospital support surface 20, from a support surface 20 to a CT Scanning Couch 30 or other combinations thereof. In these examples support surfaces could also be implemented as patient stretchers, gurneys, hospital beds, and the like.

Returning to FIGS. 2A-2C, the transfer device 100 also includes one or more conveyor belts 52 to ensure the correct kinematics during a transfer operation. The transfer device further includes a powered transport base 54 with one or more powered (or “driven”) wheels 301 able to achieve velocities in all directions and a control system 150 that in some cases may be a distributed control system 150. Optionally, the transfer device may also include additional components that further improve the user experience and device stability. Examples of optional components include, but are not limited to, one or more free-wheeling (e.g., non-powered) casters 201 on the transport device base 54 and a pedal-based central locking system 202 which has two or more positions including, for example, one which is a complete lock in all directions. In various implementations the transfer device 100 also includes one or more drive controls implemented as handlebars 203 or user-interactive rails (e.g., patient side rails) that sense the presence of a user (e.g., touching the surfaces) and/or sense forces that the user is exerting on the patient transfer device in an effort to perform transfer and transportation actions.

Various implementations of the disclosed technology provide patient transport devices 100 that include multiple features, components, subsystems, and architectures. As noted above, in various cases the transport device 100 may be a patient transportation device that is also a patient transfer device, though this is not required, and in alternative embodiments, the patient transport device 100 can be implemented as a wheeled bed, gurney, stretcher, or be provided with another desirable form. Also described elsewhere herein, various implementations of the transport device 100 include a control system 150, a human-machine interface with one or more drive controls (e.g., handlebars 203), and one or more driven wheels 301.

In some cases the control system 150 is a distributed control system 150 including a system processor and one or more subsystems and corresponding subsystem controllers. In some cases the HMI, or at least a sensor portion of the HMI controls and/or drive controls, are considered part of the control system 150. The HMI may or may not be connected with a dedicated subsystem controller. Whether incorporating a dedicated subsystem controller, a main controller, or a system processor 1110, the control system 150 receives a user input from the user via one or more drive controls to determine the direction the user wishes to transport the device. The controller/system processor is configured to interpret the users' force inputs on the drive control(s) and convert these signals to a velocity vector for the drive wheel(s) to generate in order to perform a transportation movement. In some cases the control system 150 and methods that classify and convert the user's input into commands for the driven wheel(s) 301 can be made more intuitive and accurate by using a machine learning algorithm which is described in more detail later in this disclosure.

In various implementations the drive controls are configured as handlebars 203 intended for users to grab. In various implementations, the grab bars, handlebars, or other components include force sensors 402 or load cells 401, for example, incorporated into the handlebar 203 structural mounts. Those familiar with force sensing techniques will readily appreciate that there are many other ways that force sensing can be included, such as surface mounted force sensors 403 that can be adhered to the handlebars 203 surface. FIGS. 5E-5H illustrate some examples of force sensors 402 and surface mounted force sensors 403 according to various implementations. The handlebars 203 also optionally incorporate a presence detection sensor 404 which can inform the control system 150 when the user is actively touching the handles. This presence detection sensor 404 can be used to ensure the transport device 100 is only activated to perform a transportation maneuver when the user is grabbing the handlebars 203. The presence detection sensor 404 can also prevent inadvertent bumps or other inanimate objects from exerting forces on the handlebars 203 to activate the system.

In various implementations the patient transport device 100 includes one or more additional technologies to further enhance the user experience and increase safety against collisions. Examples of possible additional technologies include, for example, imaging systems, mapping systems, and obstacle detecting systems. In some cases one or more of these additional technologies can be incorporated to reduce the likelihood of collisions and navigate during driving. For example, in various implementations the patient transport device 100 may include an ultrasonic or camera-based imaging system which would automatically brake or steer the vehicle in the event of potential collisions or obstacles. In some cases the transport device can include a GPS mapping feature in order to aid the user in navigating from one location to another. Further, in various cases LiDAR or Radar can be incorporated to improve the users' perception of local obstacles as well as to enable semi- or fully autonomous navigation of the transport device 100 when the user is unavailable or preoccupied with other tasks.

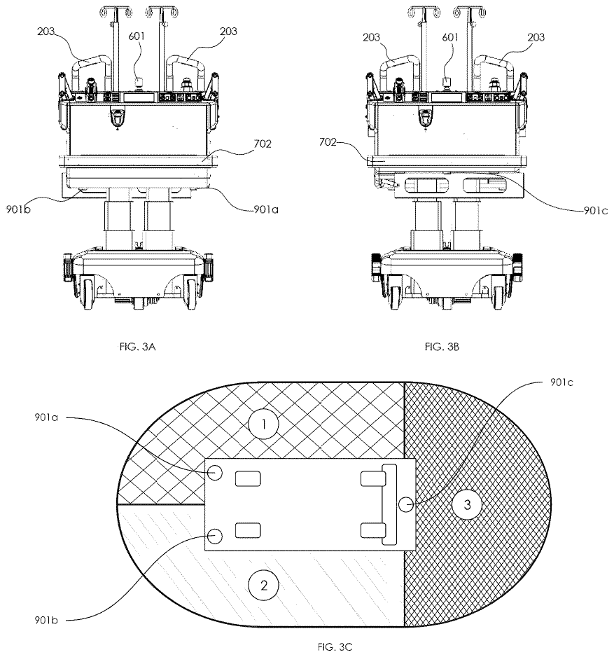

FIGS. 3A-3C illustrate an example of a LIDAR sensor array on a patient transportation device according to various implementations. In this example, three LiDAR sensors (901a-c) are arranged in such a fashion that internal components of the patient transfer device do not cause a shadow in the 360-degree field of view required to sense objects around the device. Each LiDAR sensor is configured to scan its respective zone (three zones are depicted) to provide collision avoidance capabilities to the control system. As shown in FIGS. 3A-3C, in various implementations two LiDAR sensors 901a-b are positioned at opposite corners of one end of the transportation device, while the third LiDAR sensor 901c is centered at the opposite end of the transportation device. Other sensor arrangements are also possible.

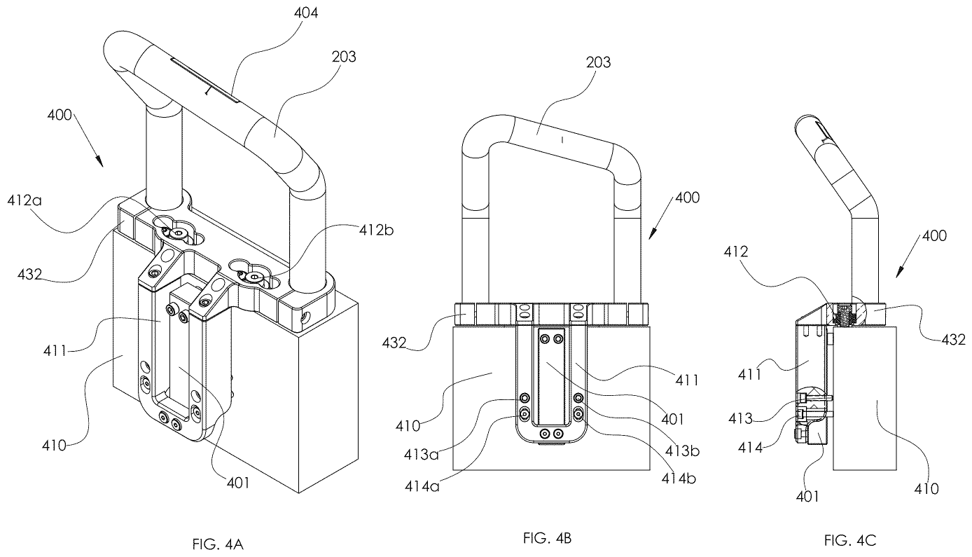

FIG. 4A-4C are schematic and isometric views of an example of a compact handlebar 203 which is mounted to the support structure 410 of a transportation device, such as a carrying structure or patient support structure. In the depicted example, the handlebar 203 is mounted with a handle mount assembly 400 that includes a mounting plate 432 connected to a lever arm 411. The handle mount assembly 400 enables forces to be sensed in both the push and pull directions via a force sensor 401, which in this case is implemented as a load cell. The force sensor 402 is affixed advantageously between the support structure 410 and the handlebar lever arm 411. The handlebar 203 mounting plate 432 is mounted on swivel joints 412 such that push and pull forces on the handlebar 203 are converted into a torque about the swivel joints, which simultaneously transfers the torque back into a direct force on the load cell 401. Hard-stop screws 413 and 414 limit the push and pull force, respectively, translated through the mount assembly in order to protect the force sensor 401. The handlebar 203 also includes touch sensors 404 for sensing the presence of a user as discussed above.

FIGS. 5A-5H illustrate various aspects of one or more force sensors incorporated into a handlebar 203, which is one example of a drive controller according to various implementations. In particular, FIGS. 5A-5B and 5C-5D depict force sensors incorporated into the handlebar 203 as load cells 401 that are pre-assembled separately and mechanically assembled to the handlebar 203 mechanical mounting structure according to various implementations. FIGS. 5E-5F depict force sensors in the form of multiple strain gauges 402 that can be affixed directly to the handlebar 203 itself, thereby achieving a more direct response and compact assembly. FIGS. 5G-5H depict force sensors in the form of capacitive or resistive force sensors 403 that are applied directly in the location of the user's grip area on the drive control.

FIG. 6 illustrates a force sensitive bumper assembly that gives feedback about the detection and magnitude of a collision. The force sensors 801a and 801b shown in this implementation of a force sensitive bumper assembly 700 are attached to a metal structural bumper 703 and 701. An elastomer or otherwise malleable force absorption bumper 702 envelopes the sensors 801a and 801b for protection. It should be appreciated that such a collision detecting bumper may be implemented with various sensors such as a tube with pneumatic (pressure) sensing, force sensitive resistors, force sensitive capacitors, or other such physical transducers.

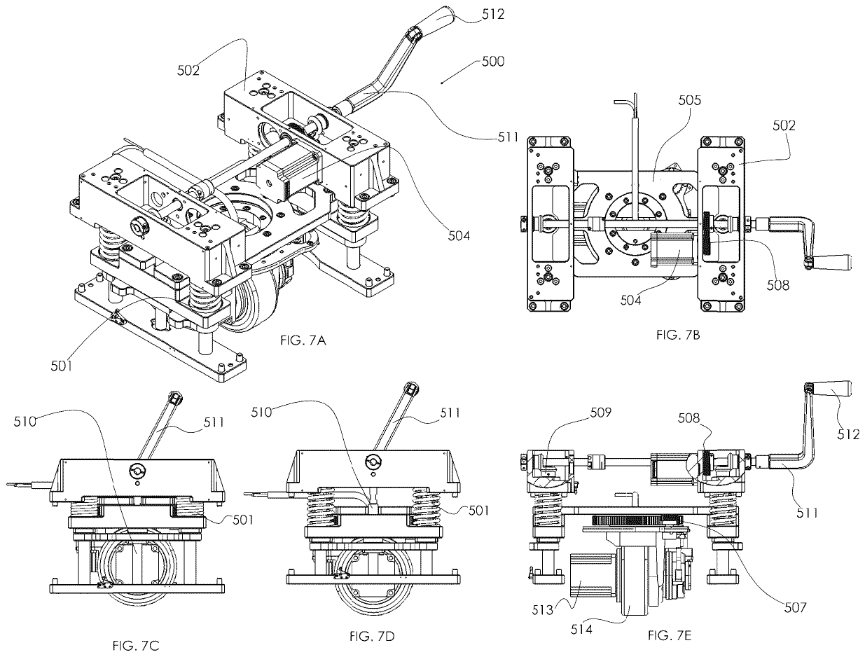

FIG. 7A-7E are views of a vertical actuation mechanism 500 useful for raising and lowering a driven transportation wheel 514 according to various implementations. The vertical actuation mechanism 500 enables vertical movement of the driven transportation wheel 514 to engage/disengage the transportation device powered wheels with/from the ground.

In various implementations, vertical actuation mechanism 500 can be powered in one or more ways. For example, in various implementations the mechanism is alternatively powered by a stepper motor 513 and manual user input to a hand crank 512. In some cases, the vertical movement is achieved through the controlled actuation of the motor 504 in order to turn gears 508 and 509 that are part of a drivetrain assembly 502. This system turns a lead screw 510 coupled with a wheel mounting bracket 505. Four springs 501 provide downward force and energize the traction of the driven transport wheel 514 when the lifting plate 501 is in its lowered position.

The hand crank 512 pictured in FIG. 7A-7E includes a main crank body 511 and a crank handle 512. The multi-part hand crank allows the vertical actuation mechanism 500 to be moved by hand into a lowered position in which the driven wheel is engaged with the ground and a raised position in which the driven wheel is disengaged from the ground. This is advantageous in situations where there is no energy source available (e.g., a dead battery) and the user must resort to disabling the transportation assistance functions manually to avoid the transportation device from physically wheeling around. It should be appreciated that this is one example of a dually energized driven wheel lifting mechanism and other mechanical arrangements are also possible and can also achieve a similar result.

According to various implementations, the transport device 100 includes one or more driven wheels 301 and, optionally, one or more non-driven wheels 201. The driven wheel(s) 301 are configured to provide locomotive force to the transport device 100 in order to achieve the desired velocity vector (speed and direction) requested by the control system 150. The drive wheels' 301 functionality may be achieved by using a multitude of drive architectures, motors and wheels, a few examples of which will be presented further in the disclosure. Various implementations may also include free-wheeling (non-driven) wheels 201 (e.g., caster wheels) that provide the transport device 100 with increased stability. In some cases the non-driven wheels 201 are able to pivot in any direction and may also include a cam-lock enabled mechanical braking mechanism.

In various cases the transport device 100 includes a foot pedal braking system 202 with a linkage that connects to braking mechanisms of one or more non-powered wheels. In some cases the braking system 202 includes a linkage that connects four pedals, one on each of the four corners of the transport device 100. In some cases a pedal will have at least two positions, but may also have three positions or more. In some cases one position corresponds to fully braked (both mechanically and electronically through the control system) and other positions inform the wheels (e.g., both driven 301 and non-driven 201) what driving mode to operate in. For example, in some cases a neutral or ‘flat’ pedal position indicates low-speed, high degree of freedom maneuvers such as within close quarters of a patient's room. This mode would also enable the ability for the transport device 100 to pivot in place and perform strafing (also known as ‘crab walking’) maneuvers. In some cases a forward pedal position (reverse being full stopped) allows for high-speed transportation actions that will have a larger turning radius (relative to the neutral pedal position) and would not allow for pivoting in place or strafing maneuvers.

According to various implementations, in the event of an upwards incline, the control method can increase the velocity vector and, in a decline, it can automatically apply a deceleration vector. In various cases this approach can be automated through the use of an inclinometer or angle sensor incorporated into the control system 150.

In various cases the control system 150 is configured with a feedback loop that enables the user to make adjustments in real time, during the act of transporting the device 100. In some cases the control method can adjust the control configuration whether there is a patient onboard the device or not. In some cases the control method monitors the user's force inputs to the handles or grab bars constantly, thereby being able to immediately adjust to the user's desired velocity vector. This control method and system approach reduces the amount of physical effort required by the operator, thereby reducing the likelihood of sustaining an injury. The control method and system also creates a more dynamic and intuitive powered transportation assistance feeling since the steering and maneuvering operations will have faster response times. This in turn can make transportation operations safer for both the operator and the patient 10.

As discussed elsewhere herein, the term “powered transportation assistant” or “powered transport assistant” is sometimes used herein to refer to a system operable to aid an operator of a patient or other healthcare facility transportation device in the act of transporting the device 100 from one location to another. In various implementations the powered transportation assistant includes multiple components and/or subsystems that work together to provide the functionality of the powered transportation assistant. For example, in some cases the powered transportation assistant incorporates aspects of one or more drive controls, the structural frame of the transport device, one or more powered wheels 301 and/or non-powered wheels 201, the transport device's control system 150, and/or particular configurations of the control system 150 that implement a machine learning algorithm that uses movement signals resulting from manipulation of a drive control to generate a velocity vector for the transport base.

According to various implementations, the powered transportation assistant is implemented as a part of a healthcare facility transportation device 100. In various implementations, the powered transportation assistant is implemented as a part of a patient transport device 100. In some cases the transport device 100 may also be a patient transfer device. In various cases a powered transport base 54 is configured to implement the powered transportation assistant with one or more drive controls that can optionally be provided separately from the base, such as forming part of the carrying structure or patient support structure of the transport device 100.

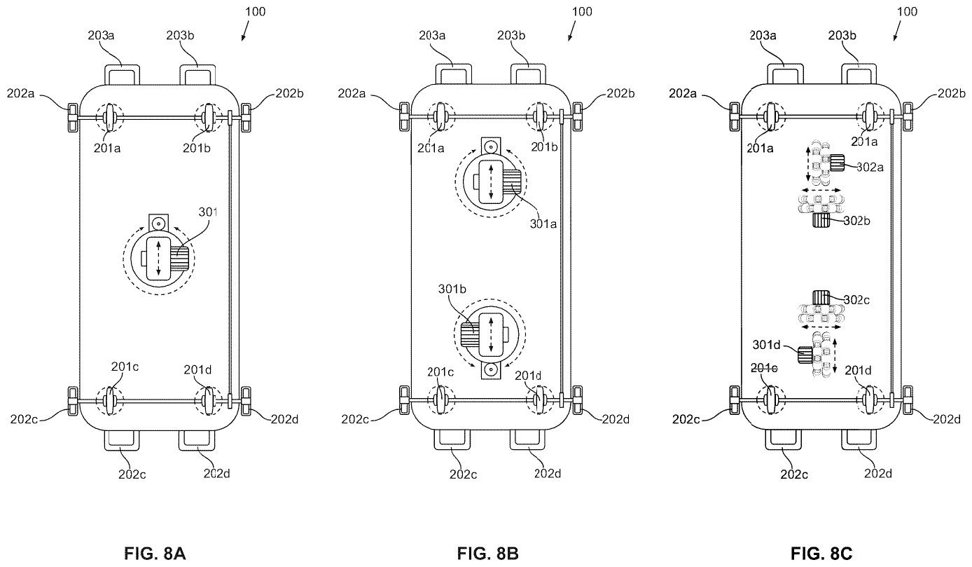

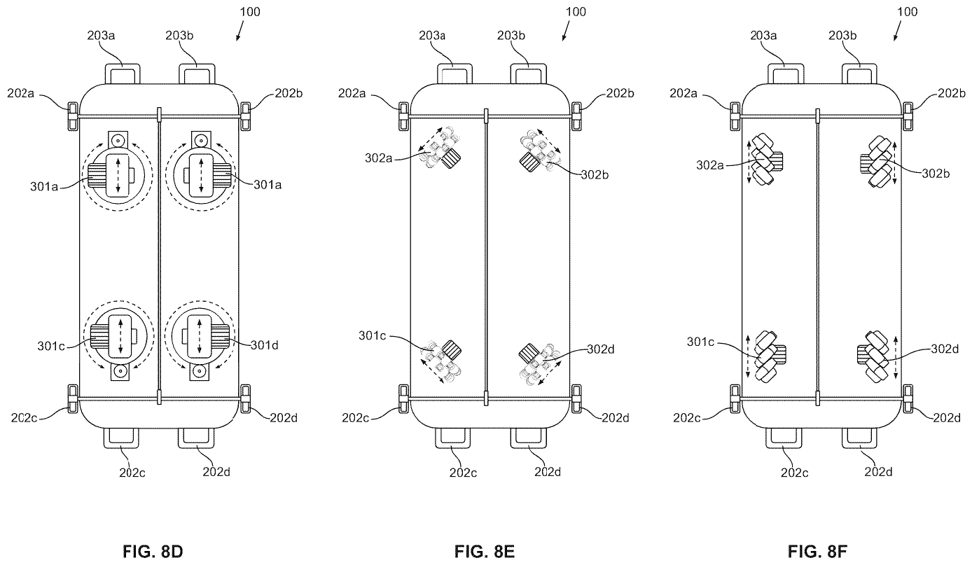

Turning to FIGS. 8-10, additional aspects and features of various implementations of healthcare facility and/or patient transport devices, systems, and/or methods will now be discussed.

FIGS. 8A-8F are schematic bottom views of various configurations of powered wheels and non-powered wheels of a patient transport device 100 according to various implementations. FIG. 8A illustrates a possible implementation in which a single powered wheel 301a is able to drive in forward and reverse directions and has a pivot proximate about its vertical axis, allowing it to drive in all directions. FIG. 8B illustrates an architecture with a pair of powered wheels 301a and 301b which both are able to drive in forward and reverse directions according to various implementations. In some cases each powered wheel is also mounted to the transport base with a slew drive that enables them to pivot about their vertical axis. Such an arrangement can provide a more stable system, and can dynamically change its turning radius in addition to pivoting and ‘crab walking.’ Accordingly, this architecture provides an improvement over the example with a single powered wheel of FIG. 8A.

FIG. 8C illustrates various implementations that may include a combination of non-driven wheels for stability as well as omnidirectional powered wheels that are driven. In some cases the driven omnidirectional wheels can be either 2-axis drive systems such as those in FIGS. 8A and FIG. 8B or they can be omnidirectional wheels such as omniwheels or mecanum wheels. FIGS. 8D and 8F illustrate examples of architectures incorporating four independent omnidirectional wheels according to various implementations.

According to various implementations, the powered wheels 301 illustrated in FIGS. 8A-8F and elsewhere herein may have a variety of architectures. In various cases the powered wheels designed for a patient transportation device 100 are generally able to generate a velocity vector in any user requested direction and within a reasonable maximum safe speed (e.g., brisk walk ~1.4 m/s), including some additional provision for management of inclines.

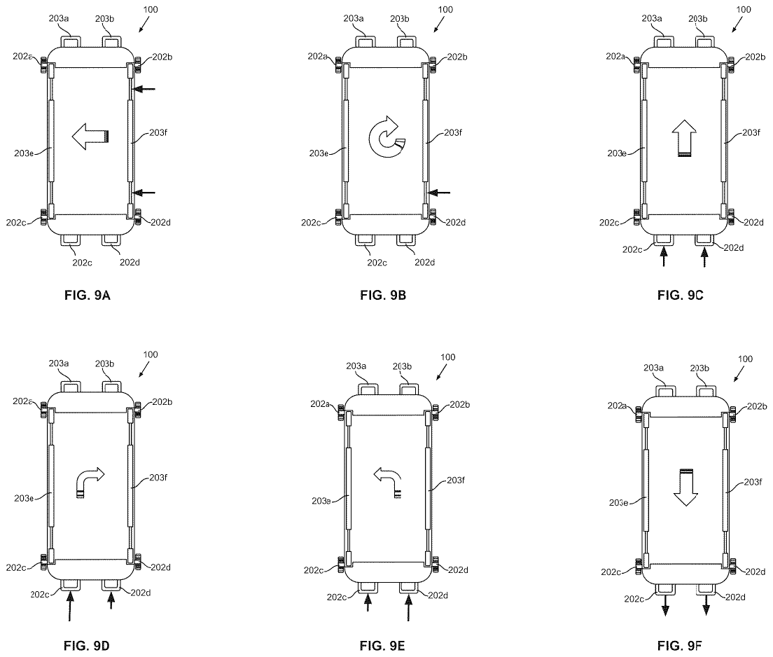

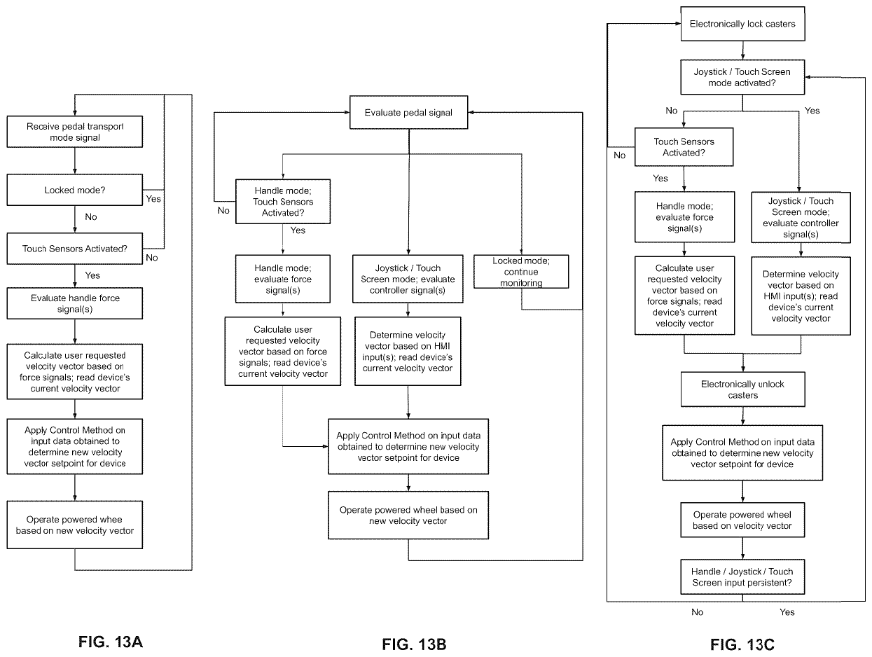

As discussed elsewhere herein, in various implementations the position of one or more of the pedals 202 can be changed to engage a corresponding feature and/or functionality of the transportation device such as one or more driving modes. It should be appreciated that in various implementations one or more of these functionalities (e.g., those represented in FIGS. 8A-8F and FIGS. 9A-9F) can be achieved without pedals 202 by replacing pedal input with an input from an alternative drive control or other HMI system. For example, in some cases an HMI touch screen can be used to engage different driving modes. Further details of such a control method are presented in FIG. 13C.

In various implementations the addition of an encoder on one or more non-driven wheels and/or an inertial measurement unit (IMU) incorporated into the distributed control system 150 can be used as closed loop feedback between a user's input at one or more drive controls (e.g., handlebars 203) and the drive wheels 301 to monitor torque in order to assess traction control. In some cases an IMU and/or encoder on non-driven wheels can also be used as a training input for the machine learning algorithm as discussed elsewhere herein.

Turning to FIGS. 9A-9F, schematic views of the relationships between various user-input forces and corresponding velocity vectors are illustrated according to various implementations. As shown in the drawings, the solid black arrows indicate forces that have been sensed by the transport device and then interpreted and used to generate a corresponding velocity vector, shown in each figure as a larger outlined arrow.

According to various implementations the patient transport device 100 includes a control system 150 that includes a system processor 1110, and optionally an additional Transport Subsystem Controller 3123, and/or other subsystem controllers 1120 that are configured to control operation of the patient transport device 100 to provide powered transportation assistance.

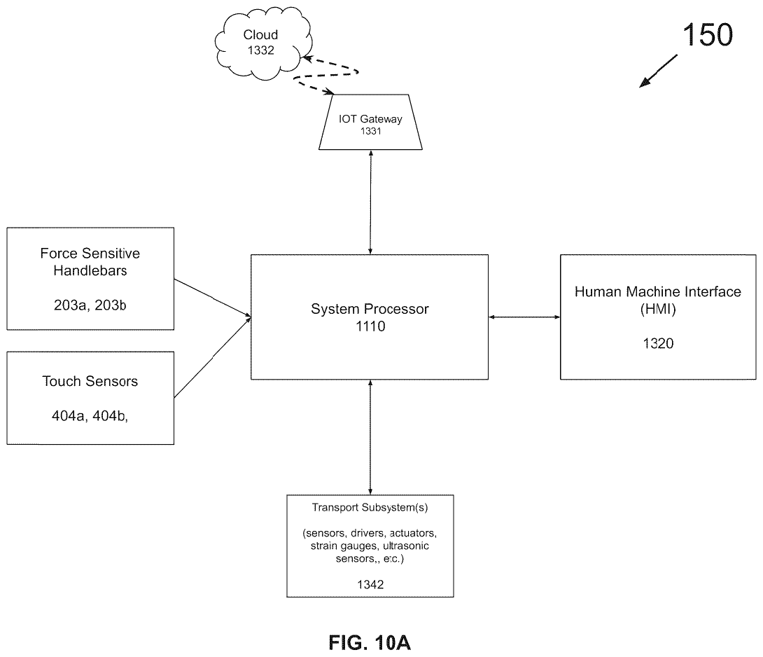

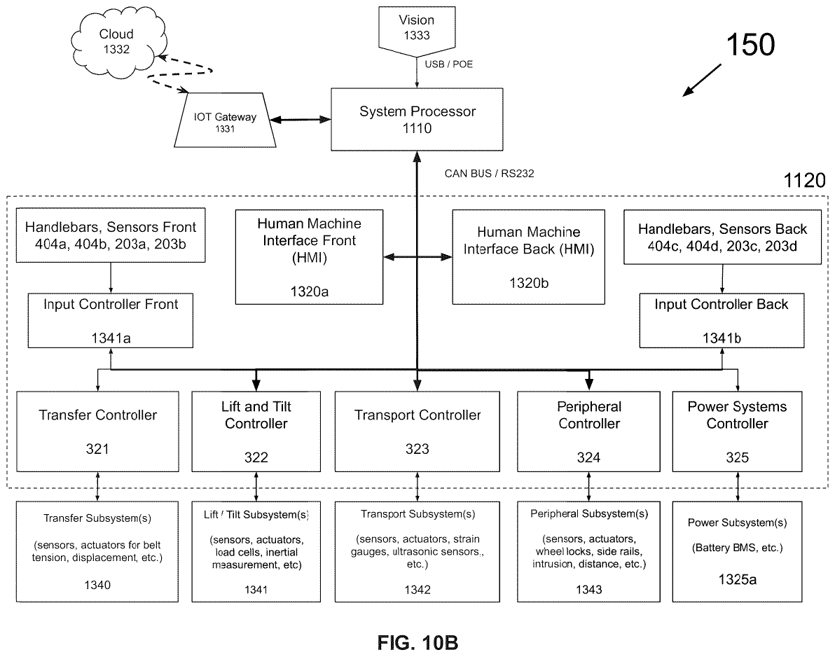

FIGS. 10A-10B are block diagrams illustrating examples of control systems for a healthcare facility transportation device according to various implementations.

FIG. 10A depicts a single-processor example control system 150 which could be used in lower cost applications such as linen carts, nutrition carts or medication carts for example. In this example, there is only a single system processor 1110 that accepts user inputs directly from a single HMI device 1320 as well as other input devices such as drive controls like force sensitive handlebars 203 and touch sensors 404.

In another example the control system 150 forms part of a patient transfer device, and is thus configured to control operation of a device transfer platform 50 through, e.g., a transfer subsystem controller 1321, as well as to provide powered transportation assistance. In various examples the control system 150 itself is configured with methods for training and improving the operation of the control system 150, including operation of the powered transportation assistant.

As shown in FIG. 10B, in various implementations the control system 150 is a distributed control system 150 including a system processor 1110 and one or more subsystems and corresponding subsystem controllers 1120. In various implementations the control system 150 includes one or more human machine interface devices (HMIs) 1320, which may include displays and/or touch screens (e.g., HMIs 1320a, 1320b). The control system 150 further includes one or more drive controls for controlling the powered transportation assistant (e.g., front/back handlebar HMIs 203a, 203b).

As shown in FIG. 10B, in some cases the system processor 1110 communicates with multiple subsystem controllers 1120. The system processor 1110 can be implemented by a variety of hardware, software and/or firmware in various cases. In some cases the system processor 1110 is configured with instructions for carrying out one or more activities and may also be referred to as a manager system. Subsystem controllers 1120 may be implemented by a variety of computing devices (e.g., one or more processors, controllers, and/or memory devices with instructions for configuring a processor or controller) which meet the requirements of various functions of the transport device and are further described elsewhere herein. The system processor 1110 may communicate with a variety of subsystem controllers 1120 depending on a particular implementation. An example of hardware utilized for the duties of the subsystem controllers may be Microprocessors or Microcontrollers with Reduced Instruction Set Computer (RISC) architecture. Depending on the requirements, the computers may have various processing power, and have commonly available data bus configurations such as 8-bit through 64-bit architectures.

FIG. 10B illustrates one possible example in which the control system 150 includes a system processor 1110 and multiple subsystem controllers 1120 that interface with and control one or more operational subsystems of the transport device. It should be appreciated that while this example illustrates a particular number of subsystem controllers 1120, implementations of the control system 150 may have any number of suitable subsystem controllers 1120. Further, implementations may include some, all, or none of the specific examples shown in FIG. 10B.

In some implementations, one of the subsystem controllers 1120 is a Transport Controller 1323. In various implementations the Transport Controller 1323 is configured to enable the user to drive the transport device 100 from one location or room to another. Such an operation can use one or more Transport subsystem(s) 1342 such as one or more driven wheels that accepts inputs for acceleration, steering, and braking. In various cases user input is received by one or more of the HMI controls and communicated to the Transport Controller 1323 by a manager system implemented by the system processor 1110 configured with appropriate instructions. In some implementations, the system processor 1110 determines the best path to transport the patient transport device 100 or intervenes on behalf of the user in order to maintain safety or automate the transportation of the transport device through direct communication to the Transport Controller 1323. According to various implementations, the system processor 1110, the Transport Subsystem Controller 3123, and/or other subsystem controllers 1120 are configured to work together to control various aspects of the powered transportation assistant.

According to various implementations, the system processor 1110 includes or is coupled with one or more physical, non-transitory computer accessible or readable storage devices, which are also referred to herein as “memory” and “memory devices.” The memory may be implemented using any suitable memory technology, which may include, e.g., temporary, and more long-term configurations, volatile and non-volatile configurations, and solid state and/or other physical formats. Examples of possible memory include random access memory (RAM), dynamic random-access memory (DRAM), static random-access memory (SRAM), magnetic hard discs, optical discs, floppy discs, flash memory, forms of electrically programmable memory (EPROM) and electrically erasable and programmable (EEPROM) memory, and other forms known in the art.

The memory device(s) coupled with the system processor 1110 contain instructions for configuring the system processor 1110 to perform particular operations or actions by virtue of loading and executing the instructions. The system processor 1110 carrying out the instructions causes the control system 150, the transportation device 100, the powered transportation assistant, and/or various training systems to carry out the desired actions. References herein to the system processor 1110 carrying out various activities imply that the system processor 1110 is configured with corresponding instructions for execution. In some cases the term “manager system” is used herein for convenience to refer to the system processor 1110 and various instructions stored in memory and/or the system processor actively operating according to various instructions (e.g., a collection of software applications stored in memory and running on the system processor).

In various implementations, the manager system collects and interprets information from user inputs and the subsystem controllers 1120 to perform analyses and calculations and automatically make decisions and subsequently coordinate and command actions for the subsystem controllers 1120. In some implementations, the manager system collects information from subsystem controllers 1120 and communicates the information to other subsystem controllers 1120. In various implementations, the manager system monitors the status and performance of the subsystem controllers 1120.

According to various implementations the control system 150 can be configured with a variety of suitable architectures which are not limited to those disclosed herein. In some cases the control system 150 may be implemented as a distributed control system as shown in FIG. 10B. Additional examples of suitable control systems 150 that may be adapted for use in accordance with the present disclosure are described in International Patent Application PCT/CA2023/050432, filed 30 Mar. 2023, titled “Transfer Device and Control System”, which is hereby incorporated herein by reference in its entirety.

According to various implementations, the control system 150 is configured with an algorithmic control method for generating a new velocity vector for driving the transportation device. In various cases the control system 150 is configured to generate the new velocity vector based on the device's current velocity vector and an operator's use of one or more drive controls. As previously noted, a velocity vector for the transportation device includes the magnitude of the velocity vector (e.g., speed) and the direction of the velocity vector (e.g., angle).

According to various implementations, the control method can be deterministic and predefined. For example, the control method may incorporate a basic control scheme such as a finite-state machine (FSM). Various additional implementations of the control system may implement an advanced control method including, for example, a machine learning model as discussed elsewhere herein.

Various implementations including a FSM control method include looking up predetermined augmentation variables based on the current velocity of the transportation device 100. In some cases the augmentation variables include sets of two variables such as a magnitude of velocity augmentation variable and an angle augmentation variable. The magnitude of velocity augmentation variable is also referred to herein as the speed augmentation variable. The method also includes hardcoding a series of finite states based on predetermined augmentation variables. In some cases the states may be stored in read only memory while programming the control system.

After looking up the predetermined speed and angle augmentation variables, the method includes calculating a resulting command vector by multiplying the augmentation variables by the current velocity vector. The resulting command vector thus includes an angle and a speed that are based on the current velocity and augmentation values corresponding to the operator's desired or intended velocity, as indicated by the operator's use of the drive control(s).

Below is an example of such a finite-state machine combined with a lookup table for augmentation variables:

$${Speed}_{new} = {Speed}_{current} + User Input Force {Magnitude}_{requested} \times Speed Augmentation Variable ( {Speed}_{current} )$$

$$Steering {Angle}_{new} = Steering {Angle}_{current} + User Input Force {Angle}_{requested} \times Steering Augmentation Variable ( {Speed}_{current} )$$

Augmentation Variable Lookup Table

| | Speed | Steering |
|---|---|---|
| Speed current | Augmentation | Augmentation |
| [m/s] | Variable | Variable |
| Greater than 1 | 0 | 0.8 |
| 0.5 to 1 | 1.25 | 1.0 |
| 0.1 to 0.5 | 1.5 | 1.1 |
| 0 | 2 | 1.25 |
| −0.1 to −0.5 | 0.75 | 1.1 |
| −0.5 to −1 | 0.25 | 1.0 |
| Less than −1 | 0 | 0.8 |

In the example above, the behavior of the FSM would result in a control method in which the transportation device achieves a maximum speed of 1 m/s in the forward direction (e.g., the operator pushing with any higher force above this speed results in no change in speed). In addition, the steering response to differences in handlebar inputs would become damped or reduced as the operator approaches the maximum pre-programmed speed. Various implementations can further include more advanced approaches to generating a control method, as is discussed elsewhere herein.

Various implementations of the technology provide training methods and systems that configure the control system 150 and methods for various examples of the patient transport device 100 described herein. In some cases the training systems and methods incorporate one or more machine learning (ML) training methods and resulting ML based control systems 150 and algorithms. In some cases a method for training an ML classification system and control method can help provide an intuitive user interaction between familiar device features such as passive handlebars and the powered transport assistant. The application of this training method and the resulting control system 150, method, and/or algorithm ensures that an appropriate and accurate velocity vector (i.e., direction and magnitude or speed) are correctly commanded to the control system to provide powered transport assistance. In some cases, higher accuracy control algorithms can result in a powered transportation assistant that will feel more intuitive and familiar to users.

Implementations of the technology provide various methods for training machine learning algorithms to classify forces applied to drive controls in relation to a resulting velocity vector for moving the patient transport device 100. In some cases the drive controls include one or more handlebars or grab and push surfaces that an operator will be interacting with. The drive controls can include one or more load sensors that generate corresponding movement signals representing the forces applied by the device operator.

Thus, in some cases a machine learning model is used to generate a control algorithm for the powered wheel(s), and also to configure the control algorithm to be more intuitive and highly assistive for the user moving the patient transfer device 100. In various implementations, the machine learning algorithm or model can be trained using an unpowered version of the patient transport device that has similar or nearly identical properties in terms of the available HMI controls, inertial properties, and wheel configurations.

In various cases the training results in a control algorithm that is not only more accurate, but also more predictable, and as a result has a reduced and/or little-to-no learning curve as well as being safer and more enjoyable to use. In comparison to classical physics modeling and computer simulation, various implementations of the disclosed training systems and methods enable development of control algorithms with significantly lower levels of effort, especially due to the complexity and degrees of freedom required to solve such a simulation. In addition, implementations of various machine learning algorithms allow for the control algorithm to be more easily updated in response to changing variables of the patient transfer device (e.g., a change in the number of drive controls and/or physical arrangement). In addition, the training steps, if included within the device manufacturing or calibration steps, would also inherently include calibration of specific variations between individual load cells or force sensors.

According to various implementations, a method for training a control algorithm can be illustrated for a patient transport device 100 having two drive controls in the form of handlebars. In this example, each handlebar has four load cells mounted orthogonally to each other. It should be appreciated that this approach can be useful for training a patient transport device 100 with any multiple of two drive controls (e.g., 2+N drive controls). The approach can also be helpful for training implementations in which one or more drive controls has multiple force sensors to, for example, provide for higher signal fidelity (e.g., 6 force sensors spaced at 60 degrees to one another).

To simulate a simple transport device arrangement, two handles—one right and one left—each having four attached load sensors are affixed to a passive (non-power assisted) mobile cart. The control system conducts machine learning to analyze the sensor measurements (e.g., sometimes referred to herein as “force signals”) from each handle to predict the movement intent of the device operator. Movement intents are captured in typical use cases such as pushing the cart forward, backward, making left and right turns, pivots about the center and so forth.

In various cases four load cells on each of the two handles provide eight measurements which are directly fed as features into the ML algorithm. These features are sampled at a reasonable rate in order to assure responsiveness and desired precision (e.g., 10 Hz or faster). In addition, differential values are calculated from the four load cells in order to generate the two orthogonal force vectors for each of the two handlebars. This results in an additional four features for the ML algorithm. Finally the total force applied to the handles can be calculated through a Pythagorean sum, thus providing an additional two features for the ML algorithm. Accordingly, in various implementations a total of fourteen features can be used in the ML algorithm analysis. In practice, only marginal increases in accuracy have been demonstrated by increasing the quantity of features in classifications. For example, using eight features (e.g., the eight direct force measurements) versus the expanded set of fourteen features (e.g., including the derived additional six calculated features) only yielded an approximate 2% increase in accuracy. This implies a highly effective machine learning training method in terms of required data and calculation required to yield an effective control model.

According to various implementations, a training algorithm includes a decision tree and/or a random forest algorithm. In various cases both algorithms have been measured to be above 90% accurate in practice, thus providing a highly intuitive and predictable motion resulting from an operator's input to the drive controls (e.g., handlebars). In various implementations, control systems and methods prioritize using the proposed training method for Machine Learning classification development for control model generation, especially considering its demonstrated high rate of accuracy. In some cases other classifier algorithms can be used with the same training method to achieve similar or even better results (e.g., Support Vector Machines, Logistic Regression methods and K-Nearest Neighbor methods).

It should also be appreciated that while load cells have been presented in various implementations, various implementations can use different sensors to measure the amount of force on one or more HMI control such as handles. Examples of possible sensors include strain gauges, force sensitive resistors (FSR), and force sensitive capacitors (FSC) to name a few. In implementations using devices like FSR and FSC, which detect a directly applied force, the sensors can be placed where an operator's hands could directly interact with the sensors. For example, in some cases an advantageous solution is to incorporate any load or strain sensing devices into the mounting system of any handle or grab bars of the patient transport device 100. This can avoid the need to place sensors in all locations a staff member might use to push a transport device, which would unnecessarily increase costs and present challenges for strict cleaning and infection prevention protocols as the sensors would be in constant contact with aggressive cleaning solutions.

In various cases, it has been shown that simply controlling the sample movements (e.g., pushing forward, backward, turning right) and repeating these actions to provide multiple sets of data to the classifier are sufficient to generate highly accurate (e.g., greater than 90%) results. However, for higher accuracy and precision, in some cases the classifier may be reinforced with additional sensors (e.g., an encoder) placed on the passive rolling wheels and/or an inertial measurement unit (IMU) for example. Such devices could help rule out any human error or inconsistencies in repeatability in the data samples for each class of movement.

A further advantage of implementing a machine learning algorithm for velocity vector interpretation, as disclosed herein for various implementations, is that detailed calibration of each load cell or the overall set of sensors for specific devices is not required, thereby potentially reducing the manufacturing and quality control costs. In various cases it is highly desirable for the sensor system to require no initial factory and ongoing calibration as that would be an ongoing operating cost for the device in manufacturing.

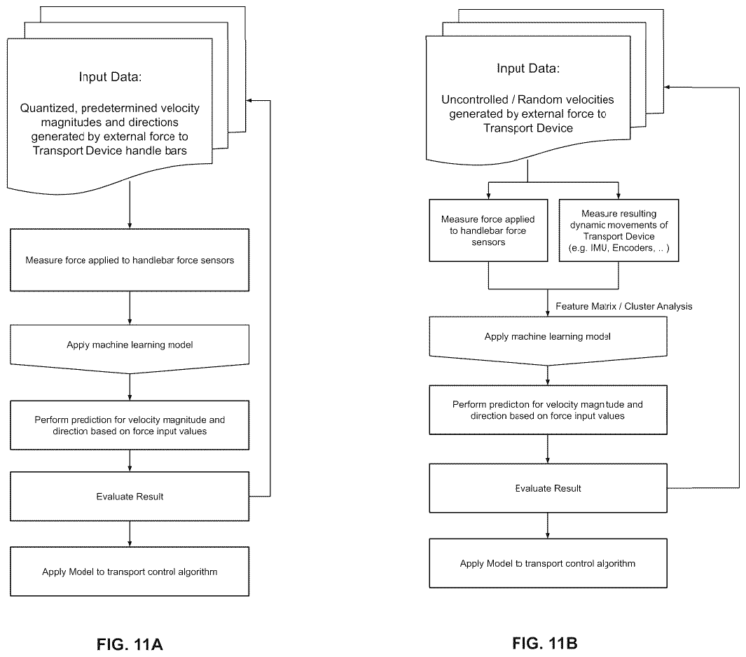

FIGS. 11A and 11B depict flow diagrams of various machine learning training approaches that can be employed to generate a control method for the control system 150 of a transportation device 100. FIG. 11A illustrates an implementation using pre-quantized and controlled data input whereby a user or fixture is instructed to exert a predetermined, controlled, and repeatable force to force sensitive handlebars 203 that have a known velocity magnitude and direction response. These quantized datasets are then applied to machine learning models in order to generate a predicted output for velocity magnitude and direction response. This predicted value is compared to the known values and evaluated for prediction accuracy. Training can continue until the predictive results are satisfactory, or alternatively additional features may be generated to improve the accuracy of the predictions. Once the results are satisfactory, a control method can be generated and incorporated into the transportation device's control system.

FIG. 11B is an example in which the transportation device is equipped with real-time closed-loop feedback for the direction and magnitude of the current velocity vector. Such data can be attained via encoders, inertial measurement units (IMUs) or similar instruments incorporated in the transportation device. Similar to the previous example, an external force is applied (that is not quantized or controlled) in various directions and at various magnitudes. The data for force and resulting velocity are both recorded at synchronized time intervals. This data can then be input to the machine learning model. Data can be collected and input to the machine learning model until satisfactory prediction accuracy is achieved. This predictive model can then be applied to the control system.

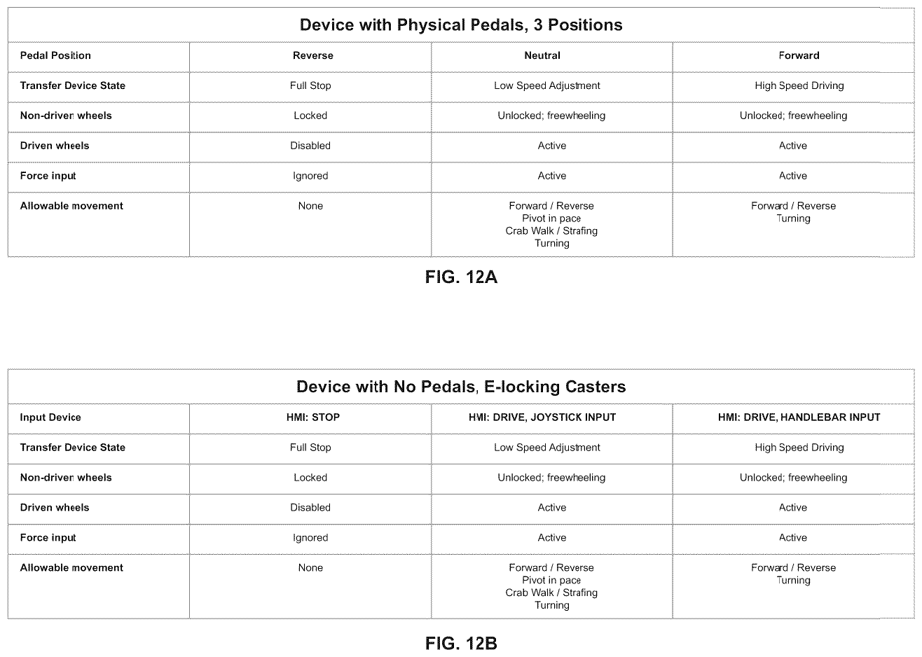

According to various implementations, the operational state of a patient transport device may depend upon one or more inputs to the control system 150. FIG. 12A illustrates one example of a state table for governing the behavior of the transport device 100 in reaction to a combination of inputs to the control system 150 according to various implementations. FIG. 12B illustrates another example of a state table for governing the behavior of a transport device 100 that includes the use of electronic braking rather than pedal brakes for casters.

FIG. 13A-C are flow diagrams that depict various examples of how a control system and process can be implemented to create intuitive powered transportation assistance as discussed herein. FIG. 13A is an example of flow diagram illustrating steps in a method for implementing a control system and process for providing powered transportation assistance which is controlled by a combination of a multi-position (locked/unlocked) foot pedal as well as touch and force sensing handlebars. In various implementations the control system/method 150 begins with:
1. The user steps on the foot pedal 202 of the transportation device 100 to indicate the transportation mode they wish to enable.
2. If necessary, all of the wheels (both driven 301 and non-driven 202) are unlocked electronically and mechanically.
3. The user grabs one of the handlebars 203 on the transportation device 100.
4. The user's presence is detected by the detection sensor 404 in the handlebar.
5. The user exerts a small force to the transportation device 100 which is sensed by the force sensor 402 incorporated into the structure of the handlebar 203.
6. The control system evaluates the current speed of the transportation device 100.
7. The input forces are measured. Using trigonometry and Pythagorean sums, the user's requested/input velocity is calculated (i.e., the magnitude and direction of velocity are calculated).
8. The user's requested velocity, along with the transportation device's current velocity are input to the control model configured in the control system 150, which generates new velocity vector.
9. If necessary, the driven wheels 301 turn or adjust to prepare to drive in the desired direction to achieve the commanded velocity vector.
10. The control system 150 continues to monitor the forces input from the user and dynamically convert these signals into velocity vectors for the driven wheels 301.
11. The user stops applying forces to the handlebars 203 and the transportation device 100 slows to a stop or the user applies a reverse force to cause the transportation device 100 to decelerate faster than normal momentum decay.
12. The user moves the foot pedal 202 into the lock position which signals to the control system 150 to turn off the transportation assistant.

FIG. 13B depicts a flow diagram for implementations of a transportation device with a control system configured to dynamically switch between pedal and joystick operation. FIG. 13C depicts a flow diagram for implementations in which the transportation device 100 may not include a physical foot pedal. In such cases the control system (e.g., HMI) is responsible for interpreting a user's inputs to decide on the appropriate drive modes based solely on the joystick, touch sensor and handlebar inputs.

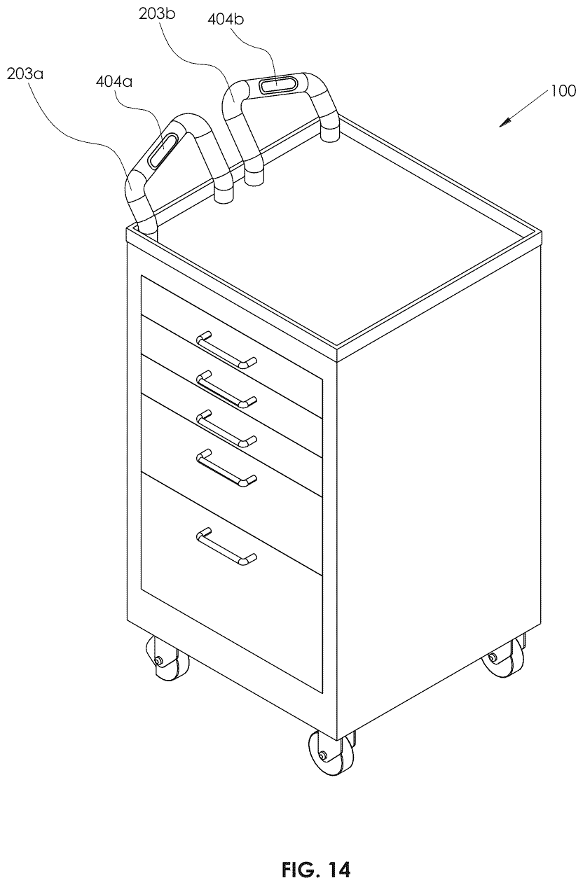

FIG. 14 depicts an example of a healthcare facility transportation device 100 implemented as a medical/medication cart utilizing two force sensitive handlebars 203.

Although the disclosure has been described with reference to certain implementations and embodiments, persons skilled in the art will recognize that changes may be made in form and detail without departing from the spirit and scope of the disclosed apparatus, systems, and methods.

What is claimed is:

1. A healthcare facility transport device, comprising:
a base comprising a plurality of wheels comprising a powered wheel;
a carrying structure mounted to the base and configured to carry at least one of healthcare supplies and a patient;
first and second handles mounted to the carrying structure;
a first force sensor mounted proximate to the first handle and configured to output a first force signal in response to sensing movement of the first handle;
a second force sensor mounted proximate to the second handle and configured to output a second force signal in response to sensing movement of the second handle; and
a control system comprising a system processor configured to:
determine an updated velocity vector, comprising:
determining a magnitude of the updated velocity vector based on values of the first and second force signals; and
determining a direction of the updated velocity vector based on the values of the first and second force signals, a first model, a current velocity of the transport device, and a distance between the first and second handles; and
operate the powered wheel based on the updated velocity vector.

2. The healthcare facility transport device of claim 1, wherein the first model comprises a first finite-state machine.

3. The healthcare facility transport device of claim 1, wherein the first model comprises a first machine learning model.

4. The healthcare facility transport device of claim 3, wherein the first machine learning model is a product of a decision tree learning algorithm.

5. The healthcare facility transport device of claim 3, wherein the first machine learning model is a product of a random forest learning algorithm.

6. The healthcare facility transport device of any one of claims 1 to 5, wherein the system processor is further configured to determine augmentation values for the updated velocity vector angle and magnitude based on the current velocity.

7. The healthcare facility transport device of any one of claims 1 to 5, wherein the system processor is further configured to determine the velocity vector based on a second machine learning model.

8. The healthcare facility transport device of any one of claims 1 to 7, further comprising a first plurality of force sensors mounted proximate to the first handle, each of the first plurality of force sensors configured to output a respective one of a first plurality of force signals in response to sensing movement of the first handle, the first plurality of force sensors comprising:
the first force sensor and a third force sensor arranged to sense movement of the first handle, respectively, in a first direction and a third direction opposite the first direction; and
fourth and fifth force sensors arranged to sense movement of the first handle, respectively, in a fourth direction and a fifth direction opposite the fourth direction; and
a second plurality of force sensors mounted proximate to the second handle, each of the second plurality of force sensors configured to output a respective one of a second plurality of force signals in response to sensing movement of the second handle, the second plurality of force sensors comprising:
the second force sensor and a sixth force sensor arranged to sense movement of the second handle, respectively, in a second direction and a sixth direction opposite the second direction; and
seventh and eighth force sensors arranged to sense movement of the second handle, respectively, in a seventh direction and an eighth direction opposite the seventh direction.

9. The healthcare facility transport device of claim 8, wherein the first plurality of force signals comprises the first force signal and third, fourth, and fifth force signals respectively corresponding to the first, third, fourth and fifth force sensors, and wherein the velocity vector is based on a Pythagorean sum and trigonometric calculation of a difference in values of the first and third force signals and a difference in values of the fourth and fifth force signals.

10. The healthcare facility transport device of claim 9, wherein the first and third directions are orthogonal with the fourth and fifth directions, and the second and sixth directions are orthogonal with the seventh and eighth directions.

11. The healthcare facility transport device of any one of claims 8 to 10, wherein the first and third force sensors are symmetrically arranged about the first handle, the fourth and fifth force sensors are symmetrically arranged about the first handle, the second and sixth force sensors are symmetrically arranged about the second handle, and the seventh and eighth force sensors are symmetrically arranged about the second handle.

12. The healthcare facility transport device of any one of claims 1 to 11, wherein the first and second handles are flexibly mounted to the carrying structure, wherein the first force sensor is connected between the first handle and the carrying structure, and wherein the second force sensor is connected between the second handle and the carrying structure.

13. The healthcare facility transport device of any one of claims 1 to 12, further comprising:
a first handle mount assembly comprising:
a first plate attached to the carrying structure;
a first lever arm connected to and extending away from the first plate; and
a second handle mount assembly comprising:
a second plate attached to the carrying structure;
a second lever arm connected to and extending away from the second plate;
wherein the first handle is connected to the first plate;
wherein the second handle is connected to the second plate;
wherein a first end of the first sensor is connected to the first lever arm and a second end of the first sensor is connected to the carrying structure; and
wherein a first end of the second sensor is connected to the second lever arm and a second end of the second sensor is connected to the carrying structure.

14. The healthcare facility transport device of any one of claims 1 to 13, wherein the system processor is further configured to augment the velocity vector based on a mass factor.

15. The healthcare facility transport device of any one of claims 1 to 14, further comprising at least one of an inertial measurement unit (IMU) and an encoder, wherein the system processor is further configured to determine a measured velocity vector of the healthcare facility transport device based on an output signal from the IMU and/or encoder.

16. The healthcare facility transport device of claim 14 or 15, wherein the system processor is configured to increase the mass factor if the velocity vector opposes the measured current velocity.

17. The healthcare facility transport device of any one of claims 14 to 16, wherein the system processor is configured to increase the mass factor to simulate decreased inertia if a magnitude of the measured velocity is below a low-speed threshold.

18. The healthcare facility transport device of any one of claims 14 to 17, wherein the system processor is configured to decrease the mass factor to simulate increased inertia if a magnitude of the measured velocity is above a high-speed threshold.

19. The healthcare facility transport device of any one of claims 1 to 18, wherein the first handle comprises a first presence sensor and the second handle comprises a second presence sensor.

20. The healthcare facility transport device of any one of claims 1 to 19, wherein the first and second handles are mounted at an end of the carrying structure.

21. The healthcare facility transport device of claim 20, wherein the first and second handles extend at least partially along a width of the carrying structure.

22. The healthcare facility transport device of any one of claims 1 to 19, wherein the first and second handles are mounted at a side of the carrying structure and extend at least partially along a length of the carrying structure.

23. The healthcare facility transport device of any one of claims 1 to 22, wherein the transport device is a medical cart.

24. The healthcare facility transport device of any one of claims 1 to 22, wherein the transport device is a patient transfer device.

25. A patient transport device, comprising:
a transport base comprising a plurality of wheels comprising a powered wheel;
a patient support structure mounted to the transport base;
a first handle mounted to the patient support structure;
a second handle mounted to the patient support structure;
a first force sensor mounted proximate to the first handle and configured to output a first force signal in response to sensing movement of the first handle, the first force signal comprising a first force value;
a second force sensor mounted proximate to the second handle and configured to output a second force signal in response to sensing movement of the second handle, the second force signal comprising a second force value; and
a distributed control system comprising a system processor and a plurality of subsystem controllers;
wherein each subsystem controller is configured to control operation of one of a plurality of subsystems of the patient transport device; and
wherein the system processor is configured to:
monitor and communicate with the plurality of subsystem controllers;
determine an updated velocity vector, comprising:
determining a magnitude of the updated velocity vector based on the first force value and the second force value; and
determining a direction of the updated velocity vector based on the first force value, the second force value, a first model, a current velocity of the transport device, and a distance between the first and second handles; and
operate the powered wheel based on the updated velocity vector.

26. The patient transport device of claim 25, further comprising:
a first sensor assembly comprising a first plurality of force sensors mounted proximate to the first handle and comprising the first force sensor, each of the first plurality of force sensors configured to output a respective one of a first plurality of force signals, comprising the first force signal, in response to sensing movement of the first handle, the first plurality of force signals comprising, respectively, a first plurality of force values comprising the first force value; and
a second sensor assembly comprising a second plurality of force sensors mounted proximate to the second handle and comprising the second force sensor, each of the second plurality of force sensors configured to output a respective one of a second plurality of force signals, comprising the second force signal, in response to sensing movement of the second handle, the second plurality of force signals comprising, respectively, a second plurality of force values comprising the second force value;
wherein the system processor is further configured to determine the magnitude of the updated velocity vector based on the first and second pluralities of force values and determine the direction of the updated velocity vector based on the first and second pluralities of force values.

27. The patient transport device of any one of claims 25 and 26, wherein the plurality of subsystems comprises a transfer subsystem comprising a transfer platform supported by the patient support structure, the transfer platform comprising a conveyor belt configured to move a patient onto and off of the transfer platform; and
wherein the plurality of subsystem controllers comprises a transfer controller configured to operate the transfer subsystem.

28. The patient transport device of any one of claims 25 to 27, wherein the plurality of subsystem controllers comprises a transport controller configured to operate the powered wheel based on the updated velocity vector.

29. The patient transport device of any one of claims 25 to 28, wherein the first model comprises a first finite-state machine.

30. The patient transport device of any one of claims 25 to 28, wherein the first model comprises a first machine learning model.

31. The patient transport device of claim 30, wherein the first machine learning model is a product of a decision tree learning algorithm.

32. The patient transport device of claim 30, wherein the first machine learning model is a product of a random forest learning algorithm.

33. The patient transport device of any one of claims 25 to 32, wherein the system processor is further configured to determine the magnitude of the updated velocity vector based on a second machine learning model.

34. The patient transport device of any one of claims 25 to 32, wherein the second machine learning model is a product of a decision tree learning algorithm or a random forest learning algorithm.

35. The patient transport device of any one of claims 25 to 34, wherein the first plurality of force values further comprises third, fourth, and fifth force values and wherein the updated velocity vector is based on a Pythagorean sum and trigonometric calculation of a difference of the first and third force values and a difference of the fourth and fifth force values.

36. The patient transport device of any one of claims 25 to 35, wherein the system processor is further configured to augment the updated velocity vector based on a mass factor.

37. The patient transport device of any one of claims 25 to 36, wherein the first and second handles are mounted at an end of the patient support structure.

38. The patient transport device of any one of claims 25 to 37, wherein the first and second handles are mounted at an end of the patient support structure and extend at least partially along a width of the patient support structure.

39. The patient transport device of any one of claims 25 to 38, wherein the first and second handles are mounted at a side of the patient support structure and extend at least partially along a length of the patient support structure.

40. The patient transport device of any one of claims 25 to 39, further comprising an angle sensor configured to sense a current pitch angle of the patient transport device, wherein the system processor is configured to adjust the updated velocity vector based on the current pitch angle so that the patient transport device decelerates on a decline and accelerates on an incline.

41. The patient transport device of any one of claims 25 to 40, further comprising a plurality of obstacle sensors configured to generate proximity measurements for obstacles near the patient transport device.

42. The patient transport device of claim 41, wherein the plurality of obstacle sensors comprises at least one of a lidar sensor and an ultrasonic sensor.

43. The patient transport device of any one of claims 25 to 42, further comprising a physical bumper sensor configured to signal physical contact with an object.

44. The patient transport device of any one of claims 25 to 43, further comprising a camera configured to generate a video stream for autonomous navigation of the patient transport device.

45. The patient transport device of any one of claims 25 to 44, wherein the system processor is configured to retrieve location data for the patient transport device and use the location data to autonomously drive the patient transport device from a first location to a second location.

46. The patient transport device of claim 45, wherein the system processor is configured to autonomously avoid obstacles while driving from the first location to the second location.

47. The patient transport device of any one of claims 26 to 46, wherein the first plurality of force sensors comprises:
the first force sensor and a third force sensor arranged to sense movement of the first handle, respectively, in a first direction and a third direction opposite the first direction; and
fourth and fifth force sensors arranged to sense movement of the first handle, respectively, in a fourth direction and a fifth direction opposite the fourth direction; and
the second plurality of force sensors comprises:
the second force sensor and a sixth force sensor arranged to sense movement of the second handle, respectively, in a second direction and a sixth direction opposite the second direction; and
seventh and eighth force sensors arranged to sense movement of the second handle, respectively, in a seventh direction and an eighth direction opposite the seventh direction.

48. The patient transport device of claim 47, wherein the first and third directions are orthogonal with the fourth and fifth directions, and the second and sixth directions are orthogonal with the seventh and eighth directions.

49. The patient transport device of any one of claims 25 to 48, wherein the first and second handles are flexibly mounted to the patient support structure, wherein the first force sensor is connected between the first handle and the patient support structure, and wherein the second force sensor is connected between the second handle and the patient support structure.

50. The patient transport device of any one of claims 22 to 49, further comprising:
a first handle mount assembly comprising:
a first plate attached to the patient support structure;
a first frame connected to and extending away from the first plate; and
a second handle mount assembly comprising:
a second plate attached to the patient support structure;
a second frame connected to and extending away from the second plate;
wherein the first handle is connected to the first plate;
wherein the second handle is connected to the second plate;
wherein a first end of the first sensor is connected to the first frame and a second end of the first sensor is connected to the patient support structure; and
wherein a first end of the second sensor is connected to the second frame and a second end of the second sensor is connected to the patient support structure.

51. A method for training a machine learning algorithm for generating an updated velocity vector for a patient transport device, the method comprising:
providing a patient transport device comprising:
a non-powered, free-wheeling transport base;
a patient support structure mounted to the transport base;
a first handle mounted to the patient support structure;
a second handle mounted to the patient support structure;
a first force sensor mounted proximate to the first handle and configured to output a first force signal in response to sensing movement of the first handle, the first force signal comprising a first force value;
a second force sensor mounted proximate to the second handle and configured to output a second force signal in response to sensing movement of the second handle, the second force signal comprising a second force value;
an inertial measurement unit (IMU); and
a control system comprising a system processor configured to determine a measured current velocity of the patient transport device based on an output signal from the IMU;
pushing and/or pulling at least one of the first handle and the second handle to move the patient transport device;
recording multiple instances of the first force value, the second force value, and the measured current velocity to generate training data; and
applying a first machine learning algorithm to the training data to produce a first machine learning model configured to predict a direction of an intended velocity vector corresponding to forces imparted to the first handle and/or the second handle.

52. The method of claim 51, further comprising applying a second machine learning algorithm to the training data to produce a second machine learning model configured to predict a magnitude of the intended velocity vector.

53. The training method of any one of claims 51 to 52, wherein at least one of the first machine learning algorithm and the second machine learning algorithm is a decision tree algorithm.

54. The training method of any one of claims 51 to 52, wherein at least one of the first machine learning algorithm and the second machine learning algorithm is a random forest algorithm.

55. A patient transport device, comprising:
a transport base comprising a plurality of wheels comprising a powered wheel;
a vertical actuation mechanism comprising a mounting bracket attached to the powered wheel, a drive train engaged with the mounting bracket, and a handle rotatably coupled with the drive train;
a patient support structure mounted to the transport base;
a first handle mounted to the patient support structure;
a second handle mounted to the patient support structure;
a first force sensor mounted proximate to the first handle and configured to output a first force signal in response to sensing movement of the first handle, the first force signal comprising a first force value;
a second force sensor mounted proximate to the second handle and configured to output a second force signal in response to sensing movement of the second handle, the second force signal comprising a second force value; and
a control system comprising a system processor configured to:
determine an updated velocity vector, comprising:
determining a magnitude of the updated velocity vector based on a sum of the first and second force values; and
determining a direction of the updated velocity vector based on the first force value, the second force value, a first model, a current velocity of the transport device, and a distance between the first and second handles; and
operate the powered wheel based on the updated velocity vector.

56. The healthcare facility transport device of claim 1, wherein the plurality of wheels comprises a plurality of powered wheels.

57. The patient transport device of claim 25, wherein the plurality of wheels comprises a plurality of powered wheels.

58. The patient transport device of claim 55, wherein the plurality of wheels comprises a plurality of powered wheels.
